(19) United States
(12) Patent Application Publication
(10) Pub. No.: US 2011/0293635 A1
(43) Pub. Date: Dec. 1, 2011

(54) COMPOSITION AND METHODS FOR MODULATING TOLL-LIKE RECEPTOR ACTIVITY

(76) Inventors: Luke Anthony John O'Neill, Dublin (IE); Susan Carpenter, Dublin (IE)

(21) Appl. No.: 13/119,116

(22) PCT Filed: Sep. 16, 2009
(86) PCT No.: PCT/EP2009/062033

§ 371 Date: Jun. 6, 2011

(30) Foreign Application Priority Data
Sep. 16, 2008 (EP) 08164464.3

(51) Int. Cl. A61K 39/395; C12N 5/071; A61K 31/713; A61K 38/02; A61K 31/7088; A61P 37/04; A61K 31/70; A61P 25/00; A61P 25/28; A61P 25/16; A61P 9/10; A61P 29/00; G01N 33/566; A61K 31/715

(52) U.S. Cl. 424/172.1; 435/7.21; 435/375; 514/44 A; 514/1.1; 514/44 R; 514/54; 514/23; 514/17.7; 514/17.8; 514/17.9

(57) ABSTRACT

The present invention relates to compositions and methods for use in the treatment of conditions such as septicaemia and septic shock. The invention further provides compositions and methods for the suppression of Toll-like Receptor 14 interaction with CD14 during Toll-like Receptor mediated signalling. The invention further provides screening assays to identify compounds which have utility in preventing the association of Toll-like Receptor 14 and CD14.

### FIELD OF THE INVENTION

The present invention relates to compositions and methods for use in modulating the association of Toll-like Receptor 14 with CD14 during Toll-like Receptor signalling. In particular, there is provided compositions comprising agents which prevent the association of Toll-like Receptor 14 with CD14 and/or which prevent the association of CD14 with a Toll-like Receptor 4 ligand. Such compositions have utility in the treatment of Toll-like Receptor 4 mediated conditions, such as sepsis, or in the treatment or prevention of diseases in which Toll-like Receptor 4 signalling is shown to have an involvement in disease progression and pathogenesis. The invention further extends to screening assays for use in identifying compounds which have utility in modulating the function of CD14.

### BACKGROUND TO THE INVENTION

The Toll-like Receptor (TLR) superfamily plays a central role in the recognition of invading pathogens and the initiation of an immune response. Each Toll-like Receptor recognises a distinct pathogen-associated molecular pattern (PAMP) leading to the activation of a signalling cascade, which in turn activates the transcription factor NF-κB and also the mitogen-activated protein kinases (MAPKs), p38, c-jun, N terminal kinase (JNK) and p42/44. Toll-like Receptor 4 (TLR-4, TLR4) also activates a further pathway which culminates in the activation of the transcription factor IFN-regulated factor-3 (IRF3), which binds to the interferon-sensitive response element (ISRE), inducing a subset of genes including interferon beta. The Toll-like Receptors are members of a larger superfamily called the interleukin-1 receptor (IL-1R)/TLR superfamily, which also contains the IL-1R1 subgroup and the TIR domain-containing adaptor subgroup. All three subgroups possess a cytoplasmic Toll/IL-1 receptor (TIR) domain, which is essential for signalling. Toll-like Receptors contain extracellular leucine rich repeats, while the IL-1R1 subgroup has extracellular immunoglobin domains. International PCT Patent Application Publication Number WO 2008/046902 teaches of a role for Toll-like Receptor 14 in LPS mediated signalling through Toll-like Receptor 4.

### SUMMARY OF THE INVENTION

The inventors have now surprisingly identified that CD14 associates with Toll-like Receptor 14 (TLR14) to form a heterodimer. The formation of this heterodimer has been identified as occurring following cellular stimulation with a Toll-like Receptor 4 ligand, such as bacterial endotoxin, for example LPS. The heterodimer complex formed between CD14 and Toll-like Receptor 14 dissociates upon the binding of a Toll-like Receptor 4 agonist to Toll-like Receptor 14. This dissociation results in an upregulation in the association between Toll-like Receptor 14 and Toll-like Receptor 4. It has also been shown that CD14 initially binds a Toll-like Receptor 4 agonist. The Toll-like Receptor 4 agonist can be transferred from CD14 to Toll-like Receptor 4 during their association in the heterodimer.

The inventors have identified that the formation of the heterodimer between TLR14 and TLR4, and the subsequent dissociation of that heterodimer can be used as the basis for assay methods for the identification of compounds which activate or suppress Toll-like Receptor 4 biological activity.

Toll-like Receptor 4 modulator agents which are identified by assay methods which use as a readout, the dissociation of the heterodimer formed between CD14 and Toll-like Receptor 14, can be used in methods for the treatment of disease conditions wherein Toll-like Receptor 4 activation contributes to disease pathology.

According to an aspect of the present invention there is provided a method for the treatment and/or prophylaxis of a disease condition which is mediated by Toll-like Receptor 4 activation and/or Toll-like Receptor 4 intracellular signalling, the method comprising the steps of:
providing a therapeutically effective amount of an agent which inhibits the dissociation of a heterodimer complex comprising Toll-like Receptor 14 and CD14, and
administering the same to a subject in need of such treatment.

Typically, the inhibition of the dissociation of the CD14/TLR14 heterodimer complex occurs when the heterodimer is complexed with a Toll-like Receptor 4 agonist compound. The Toll-like Receptor 4 agonist may be complexed with CD14 or TLR14. In certain embodiments, the Toll-like Receptor agonist is Toll-like Receptor 4.

Without wishing to be bound by theory, the inventors have identified mechanisms by which activation of Toll-like Receptor 4 (TLR4) can result, particularly where the ligand is lipopolysaccharide (LPS). Specifically, the inventors have identified a mechanism for TLR4 activation following LPS-mediated stimulation. In a first identified mechanism, the inventors predict that a heterodimer is formed from the association of Toll-like Receptor 14 and CD14. Both Toll-like Receptor 14 and CD14 are known to contain TIR domains, and as such, it is predicted that these domains allow the association of TLR14 and CD14 to form the heterodimer. Once this heterodimer has formed, it is predicted that the Toll-like Receptor 4 agonist ligand, such as LPS, initially binds to CD14. The LPS is then transferred from CD14 to Toll-like Receptor 14 (TLR14) at which time the TLR14/CD14 heterodimer complex dissociates. The LPS bound Toll-like Receptor 14 then traffics the LPS to Toll-like Receptor 4, where the LPS binds to the MD-2 adapter protein associated with TLR4. This association of LPS with MD-2 results in TLR4 activation, and in turn, intracellular signalling.

The inventors have therefore identified that there are a number of possible stages in the postulated signalling mechanism described above at which intervention by way of an antagonistic agent, or the like, can interrupt or inhibit eventual TLR4 ligand-mediated activation. For example, the inventors have identified the utility of an agent which inhibits the initial association of the TLR14/CD14 heterodimer complex.

Accordingly, in a yet further aspect of the invention, there is provided a method for treatment and/or prophylaxis of a disease condition which is mediated by Toll-like Receptor 4 activation and/or Toll-like Receptor 4 intracellular signalling, the method comprising the steps of:
providing a therapeutically effective amount of an agent which inhibits the formation of a heterodimer complex comprising Toll-like Receptor 14 and CD14, and
administering the same to a subject in need of such treatment.

The inventors have identified that a further stage at which the above pathway could be targeted, in order to suppress Toll-like Receptor 4 activation and signalling would be to prevent the initial association of the Toll-like Receptor 4 ligand, such as LPS, or a similar ligand, with CD14.

Accordingly, in a yet further aspect of the invention, there is provided a method for the treatment and/or prophylaxis of a disease condition which is mediated by Toll-like Receptor 4 activation and/or Toll-like Receptor 4 intracellular signalling, the method comprising the steps of:
providing a therapeutically effective amount of an agent which inhibits the association of a Toll-like Receptor 4 agonist ligand with CD14, and
administering the same to a subject in need of such treatment.

In certain embodiments, the Toll-like Receptor 4 activating ligand is a bacterial endotoxin, typically lipopolysaccaharide (LPS). In certain embodiments, the agent which inhibits binding of the Toll-like Receptor 4 ligand to CD14 binds to the CD14 ligand binding site, or precludes binding of a ligand to the CD14 binding site.

The inventors have identified that a yet further stage in the above described signalling pathway, which can be targeted in order to suppress Toll-like Receptor 4 activation would be to prevent the transfer of a TLR4 activating ligand from CD14 to Toll-like Receptor 14.

Accordingly, in a yet further aspect of the invention, there is provided a method for the treatment and/or prophylaxis of a disease condition which is mediated by Toll-like Receptor 4 activation and/or Toll-like Receptor 4 intracellular signalling, the method comprising the steps of:
providing a therapeutically effective amount of an agent which inhibits the transfer of a Toll-like Receptor 4 agonist ligand from CD14 to Toll-like Receptor 14, and
administering the same to a subject in need of such treatment.

In certain embodiments, the Toll-like Receptor 4 activating ligand is a bacterial endotoxin, typically lipopolysaccaharide (LPS).

The inventors have also identified that the trafficking of the Toll-like Receptor 4 agonist to Toll-like Receptor 4 may be performed directly by CD14. As such, there is not always the need for the Toll-like Receptor 4 agonist to be transferred from its initial association with CD14 to Toll-like Receptor 14.

As such, without wishing to be bound by theory, the inventors have identified that a further possible mechanism which results in TLR4 activation and TLR4-mediated intracellular signalling involves the binding of a Toll-like Receptor 4 agonist to CD14, and CD14 directly trafficking the associated agonist to the Toll-like Receptor 4 receptor complex.

Accordingly, a yet further aspect of the invention provides a method for the treatment and/or prophylaxis of a disease condition which is mediated by Toll-like Receptor 4 activation and/or Toll-like Receptor 4 intracellular signalling, the method comprising the steps of:
providing a therapeutically effective amount of an agent which inhibits the binding of a Toll-like Receptor 4 agonist ligand to CD14, and
administering the same to a subject in need of such treatment.

In certain embodiments the agent for use in any of the foregoing methods may be selected from the group consisting of, but not limited to: proteins, peptides, peptidomimetics, nucleic acids, polynucleotides, polysaccharides, oligopeptides, carbohydrates, lipids, small molecule compounds, and naturally occurring compounds. In certain embodiments, the agent is an antibody, or binding fragment derived therefrom, or a mimetic of a protein or protein fragment.

In certain embodiments, the disease condition which is mediated by Toll-like Receptor 4 activation and/or Toll-like Receptor 4 intracellular signalling is an inflammatory condition. In certain embodiments, the inflammatory condition is sepsis. In certain embodiments the Toll-like Receptor 4 agonist or Toll-like Receptor 4 activating ligand is a bacterial endotoxin. In certain embodiments, the bacterial endotoxin is lipopolysaccharide (LPS).

In certain embodiments, the disease condition which is mediated by Toll-like Receptor 4 activation and/or Toll-like Receptor 4 intracellular signalling is a neurological condition or neurodegenerative disorder. In certain embodiments, the neurodegenerative disorder or neurological condition is chosen from one or more of the group consisting of, but not limited to: Parkinson's disease, Alzheimer's disease, Huntington's disease, amyotrophic lateral sclerosis (ALS), traumatic brain injury, spinal cord injury, multiple sclerosis, ischemia or ischemia-induced injury, stroke, or a neurodegenerative condition or disorder caused by a bacterial infection. In certain embodiments, the neurodegenerative disorder or condition can be acute or chronic.

In various further aspects, the invention extends to the use of an agent in any of the foregoing methods in order to prevent the association of a Toll-like Receptor 4 ligand with CD14, to prevent the formation of a heterodimer between CD14 and Toll-like Receptor 14, or to prevent the dissociation of a heterodimer between CD14 and Toll-like Receptor 14 to prevent TLR4 activation and signalling.

Accordingly, in a yet further aspect of the invention, there is provided an agent which:
(i) inhibits the dissociation of a heterodimer complex formed between Toll-like Receptor 14 and CD14 when a Toll-like Receptor 4 agonist is bound to at least one of the Toll-like Receptor 4 or the CD14, and/or
(ii) inhibits the formation of a heterodimer complex comprising Toll-like Receptor 14 and CD14, and/or
(iii) inhibits the association of a Toll-like Receptor 4 agonist with CD14, and/or
(iv) inhibits the transfer of a Toll-like Receptor 4 agonist which is bound to CD14 to Toll-like Receptor 14, and/or
(v) inhibits the binding of a Toll-like Receptor 4 activating ligand to CD14,
(vi) inhibits the transfer of a Toll-like Receptor 4 activating ligand from CD14 to Toll-like Receptor 4,
   for use in the treatment or prevention of a disease condition which is mediated by Toll-like Receptor 4 activation and/or intracellular signalling.

A yet further aspect of the present invention provides for the use of an agent which:
(i) inhibits the dissociation of a heterodimer complex formed between Toll-like Receptor 14 and CD14 when a Toll-like Receptor 4 agonist is bound to at least one of the Toll-like Receptor 4 or the CD14, and/or
(ii) inhibits the formation of a heterodimer complex comprising Toll-like Receptor 14 and CD14, and/or
(iii) inhibits the association of a Toll-like Receptor 4 agonist with CD14, and/or
(iv) inhibits the transfer of a Toll-like Receptor 4 agonist which is bound to CD14 to Toll-like Receptor 14, and/or
(v) inhibits the binding of a Toll-like Receptor 4 activating ligand to CD14,
(vi) inhibits the transfer of a Toll-like Receptor 4 activating ligand from CD14 to Toll-like Receptor 4,
   in the preparation of a medicament for the treatment and/or prevention of a disease condition which is mediated by Toll-like Receptor 4 activation and/or intracellular signalling.

In various further aspects, the invention extends to assay methods for use in identifying agents for use in the methods of the present invention.

As such, a yet further aspect of the invention provides an assay method for the identification of an agent which inhibits Toll-like Receptor 4 activation and signalling, the method comprising the steps of:
providing a cell line expressing Toll-like Receptor 4 and exposing said cell line to a Toll-like Receptor 4 agonist,
exposing the cell line to a candidate modulator agent to determine the inhibition of Toll-like Receptor 4 activation and signalling,
observing a change in at least one cellular signalling event involving at least one of CD14, Toll-like Receptor 14 and the Toll-like Receptor 4 agonist,
   wherein at least one of:
(i) a decrease in the dissociation of a heterodimer complex comprising Toll-like Receptor 14 and CD14 in the presence of a Toll-like Receptor 4 agonist,
(ii) a decrease in the formation of a heterodimer complex comprising Toll-like Receptor 14 and CD14,
(iii) a decrease in the association of a Toll-like Receptor 4 activating ligand with CD14,
(iv) a decrease in the transfer of a Toll-like Receptor 4 activating ligand from CD14 to Toll-like Receptor 4,
(v) a decrease in the binding of a Toll-like Receptor 4 activating ligand to CD14,
(vi) a decrease in the transfer of a Toll-like Receptor 4 activating ligand from CD14 to Toll-like Receptor 14,
   indicates that the agent is a Toll-like Receptor 4 antagonist.

A yet further aspect of the invention provides a modulator agent identified by the foregoing assay methods for use in treating disease conditions which result from Toll-like Receptor 4 activation or signalling.

In various further aspects, the invention extends to assay methods for use in determining whether a compound is a Toll-like Receptor 4 agonist.

Accordingly a yet further aspect of the invention provides an assay method for the identification of a Toll-like Receptor 4 agonist, the method comprising the steps of:
providing a cell line expressing Toll-like Receptor 4,
exposing the cell line to a candidate modulator agent to determine whether the agent is an agonist of Toll-like Receptor 4 activation and signalling,
observing a change in the cellular levels of at least one of:
(i) a heterodimer comprising Toll-like Receptor 14, and CD14, and
(ii) the interaction of CD14 with Toll-like receptor 4, wherein an increase in any one of the foregoing indicates that the candidate modulator agent is a Toll-like Receptor 4 agonist.

A yet further aspect of the present invention provides an assay method for the identification of a compound which inhibits the dissociation of a heterodimer formed between Toll-like Receptor 14 and CD14, said method comprising the steps of:
providing first and second cellular samples comprising CD14, Toll-like
Receptor 4 and Toll-like Receptor 14,

contacting said first and second samples with a Toll-like Receptor 4 agonist,
contacting said first sample only with a candidate modulator agent under conditions permissive of binding of said agent to at least one of CD14 and Toll-like Receptor 14, and
monitoring the activation status of the Toll-like Receptor 4 receptor complex through a comparison of the level of downstream intracellular signalling between said first and second samples,
   wherein a reduction in Toll-like Receptor 4 signalling between said first sample and said second sample identifies the candidate modulator agent as an inhibitor of the dissociation of CD14 and Toll-like Receptor 14.

In certain embodiments, the TLR4 agonist is lipopolysaccharide (LPS).

In certain embodiments, the level of Toll-like Receptor 4 intracellular signalling is determined by quantifying the expression levels of markers or reporter molecules which indicative of Toll-like Receptor 4 activation and signalling. Examples of such markers or reporter molecules include, but are not limited to: IL-6 production, RANTES production, NF-kappaB activation, IkB levels, and IRF3 protein activation.

In a further aspect, the invention extends to assay methods for use in identifying modulators of Toll-like Receptor 4 activation and signalling, by means of the signalling pathway described herein. Such assays would, for example, be based upon FRET (fluorescence resonance energy transfer), a method used in the quantification of molecular dynamics in protein to protein interactions.

The functionality of the FRET (fluorescence resonance energy transfer) assay would be well known to a person skilled in the art. Briefly, in order to monitor the association of, and complex formation between, two molecules, one of the molecules is labelled with a fluorophore donor molecule, while the other is labelled with a fluorophore acceptor molecule. When the two molecules interact, the donor emission is transferred to the acceptor molecule. This results in the acceptor molecule emitting a light output that can be monitored. When the donor and acceptor are in close proximity, say 1-10 nm, the two molecules interact, with the resulting light output being monitored. The emission from the acceptor molecule is due to the intermolecular fluorescence resonance energy transfer from the donor to the acceptor molecule. Examples of fluorophore molecules used in such assays are cyan fluorescent protein (CFP) and yellow fluorescent protein (YFP).

In certain embodiments, the FRET assays described herein can be performed using the bead-based ALPHASCREEN technique (Perkin Elmer) as described in Ullman et al. PNAS, vol 91, pp 5426-5430, June 1994. The AlphaScreen assay contains two bead types, donor and acceptor beads. Beads can be coupled to the molecules of interest, interaction between the molecules captured on the beads leads to an energy transfer from one bead to the other resulting in a fluorescent/luminescent signal. Advantageously the AlphaScreen assay system permits for high throughout screening and accordingly the assay methods of the present invention which utilise FRET can be used in this format.

Advantageously the AlphaScreen assay system permits for high throughput screening and accordingly the assay methods of the present invention which utilise FRET can be used in HTS screening methods to facilitate the identification of modulator agent compounds.

In a further aspect, the invention provides an assay method for the identification of a compound which inhibits the formation of a heterodimer between Toll-like
Receptor 14 and CD14, said method comprising the steps of: providing first and second cellular samples comprising CD14, Toll-like Receptor 4 and Toll-like Receptor 14,
contacting said first and second samples with a Toll-like Receptor 4 agonist,
contacting said first sample only with a candidate modulator agent under conditions permissive of binding of said agent to at least one of CD14 and Toll-like Receptor 14, and
monitoring the activation status of the Toll-like Receptor 4 receptor complex through a comparison of the level of downstream intracellular signalling between said first and second samples,
   wherein a reduction in Toll-like Receptor 4 signalling between said first sample and said second sample identifies the candidate modulator agent as an inhibitor of the formation of a heterodimer between CD14 and Toll-like Receptor 14.

In certain embodiments, the TLR4 agonist is lipopolysaccharide (LPS). In certain embodiments the level of Toll-like Receptor 4 intracellular signalling is determined by monitoring markers indicative of Toll-like Receptor 4 activity selected from the group consisting of; NF-kappaB activation, and IRF3 protein activation.

An assay method for the identification of a compound which inhibits the binding of a Toll-like Receptor 4 agonist to CD14, said method comprising the steps of:
providing first and second cellular samples comprising CD14,
contacting said first and second samples with a Toll-like Receptor 4 agonist,
contacting said first sample only with a candidate modulator agent under conditions permissive of binding of said agent to CD14, and
monitoring the activation status of the Toll-like Receptor 4 receptor complex through a comparison of the level of downstream intracellular signalling between said first and second samples,
   wherein a reduction in Toll-like Receptor 4 signalling between said first sample and said second sample identifies the candidate modulator agent as an inhibitor of the binding of a Toll-like Receptor 4 agonist to CD14.

Accordingly in a further aspect of the present invention there is provided a method for the identification of an agent which acts as an antagonist of Toll-like Receptor 4 activation and intracellular signalling, said method comprising the steps of:
providing first and second cellular samples containing Toll-like Receptor 14 and CD14,
labelling the Toll-like Receptor 14 with a first fluorophore molecule and the CD14 with a second fluorophore molecule,
contacting said first and second samples with a Toll-like Receptor δ agonist,
contacting said first sample only with a candidate modulator agent under conditions permissive of binding of Toll-like Receptor 4 and/or Toll-like Receptor 14, and
monitoring the interaction of CD14 and TLR14 to determine any dissociation which occurs in the presence of the Toll-like Receptor 4 agonist and the candidate agent by monitoring the fluorescence of the fluorophores,
   wherein a decrease in the level of fluorescence is indicative of the candidate modulator agent not being an antagonist of Toll-like Receptor 4 activation and intracellular signalling, as dissociation of Toll-like Receptor 14/CD14 heterodimer complex is occurring.

In various embodiments, the assay methods of the invention are in-vitro assay methods.

In certain further aspects, various assays for measuring TLR4 activation and/or identifying modulators of TLR4 activation can be used. For example, a screening assay for TLR4 stimulation may use cells in culture which are transfected with two plasmids, one carrying the gene for human TLR4 and the other, a detector plasmid, carrying a promoter that binds to NFkappa B upstream of a luciferase gene. Alternatively a yeast two-hybrid system can be used for screening for TLR4 activation.

In certain further embodiments, screening assays to identify modulators of the Toll-like Receptor 4 signalling pathway, which involve CD14, may be in-vitro assays. Said in-vitro assays, examples of which would be well known to the person skilled in the art, could be configured to explore Toll-like Receptor 14-CD14 interactions in order to allow the identification of compounds that inhibit or promote heterodimer complex formation.

In certain embodiments, the assay may be a cell based assay which assesses Toll-like Receptor 4 dependent activation. In certain further aspects, a biochemical assay, examples of which would be well known to the person skilled in the art, could also be used to confirm the mechanism of action of an agent which is identified by an assay method of the invention to achieve a desired function, for example, the prevention of the dissociation of the TLR14/CD14 heterodimer complex. In certain further aspects, the assay may use, as a readout, at least one of: IL-6 production, RANTES production, TNF-alpha production, IL-1 beta release, and phosphorylation of p38.

Having identified the observed inter-relationships between CD14 and TLR14 and also between TLR4 and TLR14 which are postulated to result in LPS-mediated activation and signalling, the inventors predict, without wishing to be bound by theory, that following dissociation of the heterodimer complex formed between CD14 and TLR14, TLR14 translocates such that it interacts with TLR4. As TLR14 is associated with the Toll-like Receptor 4 agonist, such as LPS, the translocation of TLR14 within the cell results in LPS trafficking, with the LPS being brought into contact with Toll-like Receptor 4, typically through the adapter protein MD-2.

The inventors recognise that one of the main groups of Toll-like Receptor 4 agonist compounds are bacterial endotoxins, such as lipopolysaccharide (LPS). As such, the interrelationship between TLR14, CD14 and TLR4 interaction defined herein has been recognised by the inventors as having utility methods for the identification of compounds for use in the treatment of endotoxin mediated conditions, such as sepsis.

Accordingly, a yet further aspect of the present invention provides an assay for identifying compounds suitable for use in the treatment of endotoxin mediated conditions, said assay comprising the steps of:
providing a candidate modulator agent,
bringing the candidate modulator agent into contact with the TLR4 receptor complex,
bringing the TLR4 receptor complex into contact with endotoxin,
monitoring the light emission from fluorophore moieties which are conjoined to the TLR4 and to TLR14,
   wherein an increase in light emission level from the fluorophore indicates that the candidate modulator agent is not useful in the treatment of endotoxin mediates conditions as there is an increase in associate of TLR4 and TLR14 and as such, signalling through the TLR4 receptor complex is not being antagonised.

In certain embodiments the endotoxin mediated condition is sepsis or septic shock. In certain embodiments, the endotoxin is lipopolysaccharide (LPS) derived from a gram negative bacteria.

The conditions of septicaemia or septic shock are caused by endotoxin, such as lipopolysaccharide (LPS), which is derived from gram negative bacteria. In certain embodiments, the gram negative bacteria may be selected from the group comprising, but not limited to: Neisseria meningitides, Escherichia coli, Pseudomonas aeruginosa, Haemophilia influenzae, Salmonella typhimurium, and Francisella tularensis.

In making the surprise identification that the dissociation of a heterodimer formed between TLR14 and CD14 is involved in TLR4 activation and signalling, the inventors have recognised that TLR4 mediated signalling can be enhanced by promoting the dissociation of TLR14 and CD14. Such a dissociation of the interaction of TLR14 and CD14 can be mediated by a compound, which may be termed an adjuvant, and which could therefore be used to promote the onset and progression of an immune response, in particular an immune response which is driven by signalling through Toll-like Receptor 4.

Accordingly, in various further aspects, the invention extends to the use of compounds which promote the dissociation of a heterodimer formed between TLR14 and CD14 for use as an adjuvant composition for the enhancement of an immune response mediated by Toll-like Receptor 4 activation and signalling. In certain embodiments, the compound is administered as an adjuvant along with a vaccine composition, said vaccine composition mediating, at least in part, an immune response through Toll-like Receptor 4.

In various further aspects, the invention extends to assay methods for identifying compounds which promote the dissociation of the heterodimer formed between CD14 and TLR14, in order to enhance a Toll-like Receptor 4 response.

Accordingly a yet further aspect of the invention provides an assay method for the identification of an agent which promotes the dissociation of the heterodimer formed between Toll-like Receptor 14 and CD14, the method comprising the steps of:
providing a cell line expressing Toll-like Receptor 14, CD14 and Toll-like Receptor 4,
contacting said first and second samples with a Toll-like Receptor 4 agonist,
exposing the cell line to a candidate modulator agent under conditions which will allow the binding of the agent to CD14 and/or Toll-like Receptor 14 in order to determine whether the agent promotes the dissociation of the heterodimer formed between Toll-like Receptor 14 and CD14,
monitoring the activation status of the Toll-like Receptor 4 receptor through a comparison of the level of downstream intracellular signalling between said first and second samples,
   wherein an increase in Toll-like Receptor 4 signalling between said first sample and said second sample identifies the candidate modulator agent as an agent which promotes the dissociation of the heterodimer formed between Toll-like Receptor 14 and CD14.

A yet further aspect of the present invention relates to an assay method for the identification of a compound which promotes the dissociation of the heterodimer formed between CD14 and TLR14 to enhance a Toll-like Receptor 4 mediated immune response, said method comprising the steps of:
providing first and second cellular samples containing Toll-like Receptor 14 and CD14,
labelling the Toll-like Receptor 14 with a first fluorophore molecule and the CD14 with a second fluorophore molecule,
contacting said first and second samples with an agent which is a candidate modulator which promotes the dissociation of the interaction of CD14 and TLR14,
contacting said first sample only with the candidate modulator agent under conditions permissive of binding of Toll-like Receptor 4 and/or Toll-like Receptor 14, and
monitoring the interaction of CD14 and TLR14 to determine any dissociation which occurs in the presence of the modulator agent by monitoring the fluorescence of the fluorophores,
   wherein a decrease in the level of fluorescence indicates that the candidate modulator agent promotes the dissociation of the heterodimer formed between CD14 and TLR14.

In certain further embodiments, the present invention extends to an agent which inhibits the dissociation of a heterodimer complex between Toll-like Receptor 14 and CD14 for use in treating or preventing a disease condition which is mediated by Toll-like Receptor 4 activation and/or Toll-like Receptor 4 intracellular signalling.

In certain further embodiments, the present invention extends to the use of an agent which inhibits the dissociation of a heterodimer complex between Toll-like Receptor 14 and CD14 in the preparation of a medicament for the treatment or prevention of a disease condition which is mediated by Toll-like Receptor 4 activation and/or Toll-like Receptor 4 intracellular signalling.

In certain further embodiments, the present invention extends to an agent which inhibits the initial formation of a heterodimer complex between Toll-like Receptor 14 and CD14 for use in treating or preventing a disease condition which is mediated by Toll-like Receptor 4 activation and/or Toll-like Receptor 4 intracellular signalling.

In certain further embodiments, the present invention extends to the use of an agent which inhibits the initial formation of a heterodimer complex between Toll-like Receptor 14 and CD14 in the preparation of a medicament for the treatment or prevention of a disease condition which is mediated by Toll-like Receptor 4 activation and/or Toll-like Receptor 4 intracellular signalling.

In certain further embodiments, the present invention extends to an agent which inhibits the binding of a Toll-like Receptor 4 agonist compound to CD14 for use in treating or preventing a disease condition which is mediated by Toll-like Receptor 4 activation and/or Toll-like Receptor 4 intracellular signalling.

In certain further embodiments, the present invention extends to the use of an agent which inhibits the binding of a Toll-like Receptor 4 agonist compound to CD14 in the preparation of a medicament for the treatment or prevention of a disease condition which is mediated by Toll-like Receptor 4 activation and/or Toll-like Receptor 4 intracellular signalling.

In certain further embodiments, the present invention extends to an agent which prevents the translocation of a Toll-like Receptor 4 agonist which is bound to CD14 to Toll-like Receptor 14 for use in treating or preventing a disease condition which is mediated by Toll-like Receptor 4 activation and/or Toll-like Receptor 4 intracellular signalling.

In certain further embodiments, the present invention extends to the use of an agent which prevents the translocation of a Toll-like Receptor 4 agonist which is bound to CD14 to Toll-like Receptor 14 in the preparation of a medicament for the treatment or prevention of a disease condition which is mediated by Toll-like Receptor 4 activation and/or Toll-like Receptor 4 intracellular signalling.

By “a disease condition which is mediated by Toll-like Receptor 4 activation and/or Toll-like Receptor 4 intracellular signalling” it is meant a disease conditions which is mediated by a Toll-like Receptor 4 agonist, such as a bacterial endotoxin, binding to Toll-like Receptor 4, this resulting in the activation of Toll-like Receptor 4 and the triggering of an intercellular signalling cascade.

In certain embodiments, the disease condition which is mediated by Toll-like Receptor 4 activation and/or Toll-like Receptor 4 intracellular signalling is an inflammatory condition. In certain embodiments, the inflammatory condition is sepsis. In certain embodiments the Toll-like Receptor 4 agonist or Toll-like Receptor 4 activating ligand is a bacterial endotoxin. In certain embodiments, the bacterial endotoxin is lipopolysaccharide (LPS).

In certain embodiments, the disease condition which is mediated by Toll-like Receptor 4 activation and/or Toll-like Receptor 4 intracellular signalling is a neurological condition or neurodegenerative disorder. In certain embodiments, the neurodegenerative disorder or neurological condition is chosen from one or more of the group consisting of, but not limited to: Parkinson's disease, Alzheimer's disease, Huntington's disease, amyotrophic lateral sclerosis (ALS), traumatic brain injury, spinal cord injury, multiple sclerosis, ischemia or ischemia-induced injury, stroke, or a neurodegenerative condition or disorder caused by a bacterial infection. In certain embodiments, the neurodegenerative disorder or condition can be an acute or chronic disorder.

In certain embodiments, the agent is a small molecule compound. In certain further embodiments, the agent is an antibody, or a fragment thereof, or a peptide or a peptidomimetic thereof.

In certain further embodiments, the present invention extends to an agent which promotes the dissociation of a heterodimer complex between Toll-like Receptor 14 and CD14 for use in enhancing an immune response mediated by Toll-like Receptor 4 activation and signalling.

In certain further embodiments, the present invention extends to the use of an agent which enhances the dissociation of a heterodimer complex between Toll-like Receptor 14 and CD14 in the preparation of a medicament for the enhancement of an immune response mediated by Toll-like Receptor 4 activation and signalling.

In certain further aspects of the present invention, there is provided a kit for the performance of any one of the foregoing screening assays, said kit comprising the components required to perform the assay, such as an in-vitro cell line expressing both CD14 and Toll-like Receptor 14, and instructions for the use of the same.

### BRIEF DESCRIPTION OF THE FIGURES

FIG. 1 shows that endogenous TLR14 binds to TLR4 and TLR2,

FIG. 2 shows that endogenous TLR14 interacts with TLR4 when stimulated with lipopolysaccharide (LPS),

FIG. 3 shows that endogenous TLR14 interacts with CD14, this interaction decreasing following lipopolysaccharide (LPS) stimulation,

FIG. 4 shows that transient over-expression of TLR14 (KIAA0644) enhances IL-6 and RANTES production in U373 parental cells,

FIG. 5 shows 2 graphs showing transient over-expression of TLR14 (KIAA0644) in MEF cells causes an increase in RANTES production in response to both rough and smooth LPS stimulation,

FIG. 6 shows that transient over-expression of TLR14 (KIAA0644) in MEF cells causes an increase in IL-6 production in response to both rough (A) and smooth (B) LPS,

FIG. 7 shows that reconstitution of U373 parental cells in serum free media (SFA) with TLR14 (KIAA0644) boosts the LPS signalling pathway but not the TNF-α signalling pathway,

FIG. 8 shows that partial knockdown of TLR14 (KIAA0644) in U373CD14 cells affects the LPS signalling pathway,

FIG. 9 shows that knockdown of TLR14 (KIAA0644) in U373/CD14 cells does not affect the TNF-α signalling pathway,

FIG. 10 shows that TLR14 (KIAA0644) can be knocked down using siRNA in human peripheral blood mononuclear cells,

FIG. 11 shows that RT-PCR confirms knockdown of TLR14 (KIAA0644) in human PBMC,

FIG. 12 shows that knockdown of TLR14 (KIAA0644) in PBMC affects IκB degradation in response to LPS stimulation,

FIG. 13 shows that knockdown of TLR14 (KIAA0644) affects phosphorylation of p38 in response to LPS stimulation,

FIG. 14 shows that knockdown of TLR14 (KIAA0644) in PBMC causes a decrease in IL-6, TNF-α and IL-1β release in response to LPS stimulation, and

FIG. 15 shows that knockdown of TLR14 (KIAA0644) in PBMC does not affect the TNF-α signalling pathway.

### DETAILED DESCRIPTION OF THE INVENTION

The inventors have surprisingly identified the involvement of CD14 in Toll-like Receptor 4 activation and signalling. CD14 has been shown to initially associate with a Toll-like Receptor 4 agonist and have a role in the trafficking of this agonist to Toll-like Receptor 4. CD14 has further been shown to associate with Toll-like Receptor 14 to form a heterodimer complex. This heterodimer complex dissociates during the trafficking of the Toll-like Receptor 4 ligand. Accordingly, the present invention provides screening assays to identify agents which modulate either the dissociation of TLR14 with CD14 or the association of a Toll-like Receptor 4 agonist with CD14. The invention further extends to the use of agents which modulate either the dissociation of TLR14 with CD14 or the association of a Toll-like Receptor 4 agonist with CD14 for the treatment of a Toll-like Receptor 4 mediated condition.

Without wishing to be bound by theory, the inventors predict that the involvement of CD14 in endotoxin-mediated signalling results from CD14 complexing with a Toll-like Receptor 4 agonist, such as bacterial endotoxin, for example LPS. The CD14 forms a heterodimer with TLR14. The CD14 bound LPS is transferred to the TLR14 present in the heterodimer complex. The TLR14/CD14 heterodimer then dissociates with the LPS bound TLR14 trafficking the LPS to Toll-like Receptor, where Toll-like Receptor 14 acts as a co-receptor.

The inventors have further identified that Toll-like Receptor 4 activation can occur in the absence of TLR14, or where TLR14 is not involved in trafficking the TLR4 ligand to TLR4. In such instances, CD14 directly complexes the TLR4 agonist, such as LPS, and translocates to the TLR4 receptor complex, where the LPS is transferred to the TLR4 receptor complex, this resulting in TLR4 activation.

Toll-like Receptor 14 (TLR14), is a leucine rich repeat containing protein, the human form of which comprises the amino acid sequence of SEQ ID NO:1. TLR14 may also be known as leucine rich repeat containing protein KIAA0644 (Kazusa accession number AB014544, as defined in SWISS PROT/TrEMBL (www.expasy.org/www.uniprot.org) database under accession number Q7L0X0).

The amino acid sequence of human Toll-like Receptor 14 is provided below as SEQ ID NO:1:

MEAARALRLLLVVCGCLALPPLAEPVCPERCDCQHPQHLLCTNRGLRVVP
KTSSLPSPHDVLTYSLGGNFITNITAFDFHRLGQLRRLDLQYNQIRSLHP
KTFEKLSRLEELYLGNNLLQALAPGTLAPLRKLRILYANGNEISRLSRGS
FEGLESLVKLRLDGNALGALPDAVFAPLGNLLYLHLESNRIRFLGKNAFA
QLGKLRFLNLSANELQPSLRHAATFAPLRSLSSLILSANSLQHLGPRIFQ
HLPRLGLLSLRGNQLTHLAPEAFWGLEALRELRLEGNRLSQLPTALLEPL
HSLEALDLSGNELSALHPATFGHLGRLRELSLRNNALSALSGDIFAASPA
LYRLDLDGNGWTCDCRLRGLKRWMGDWHSQGRLLTVFVQCRHPPALRGKY
LDYLDDQQLQNGSCADPSPSASLTADRRRQPLPTAAGEEMTPPAGLAEEL
PPQPQLQQQGRFLAGVAWDGAARELVGNRSALRLSRRGPGLQQPSPSVAA
AAGPAPQSLDLHKKPQRGRPTRADPALAEPTPTASPGSAPSPAGDPWQRA
TKHRLGTEHQERAAQSDGGAGLPPLVSDPCDFNKFILCNLTVEAVGADSA
SVRWAVREHRSPRPLGGARFRLLFDRFGQQPKFHRFVYLPESSDSATLRE
LRGDTPYLVCVEGVLGGRVCPVAPRDHCAGLVTLPEAGSRGGVDYQLLTL
ALLTVNALLVLLALAAWASRWLRRKLRARRKGGAPVHVRHMYSTRRPLRS
MGTGVSADFSGFQSHRPRTTVCALSEADLIEFPCDRFMDSAGGGAGGSLR
REDRLLQRFAD

The amino acid sequence of the murine form of Toll-like Receptor 14 has also been defined. This is shown below as SEQ ID NO:2:

MEGVGAVRFWLVVCGCLAFPPRAESVCPERCDCQHPQHLLCTNRGLRAVP
KTSSLPSPQDVLTYSLGGNFITNITAFDFHRLGQLRRLDLQYNQIRSLHP
KTFEKLSRLEELYLGNNLLQALVPGTLAPLRKLRILYANGNEIGRLSRGS
FEGLESLVKLRLDGNVLGALPDAVFAPLGNLLYLHLESNRIRFLGKNAFS
QLGKLRFLNLSANELQPSLRHAATFVPLRSLSTLILSANSLQHLGPRVFQ
HLPRLGLLSLSGNQLTHLAPEAFWGLEALRELRLEGNRLNQLPLTLLEPL
HSLEALDLSGNELSALHPATFGHQGRLRELSLRDNALSALSGDIFAASPA
LYRLDLDGNGWTCDCRLRGLKRWMGNWHSQGRLLTVFVQCRHPPALRGKY
LDYLDDQLLQNGSCVDPSPSPTAGSRQWPLPTSSEEGMTPPAGLSQELPL
QPQPQPQQRGRLLPGVAWGGAAKELVGNRSALRLSRRGPGPHQGPSAAAP
GSAPQSLDLHEKPGRGRHTRANLSQTEPTPTSEPASGTPSARDSWQRAAK
QRLASEQQESAVQSVSGVGLPPLVSDPCDFNKFILCNLTVEAVSANSASV
RWAVREHRSPRPQGGARFRLLFDRFGQQPKFQRFVYLPERSDSATLHELR
GDTPYLVCVEGVLGGRVCPVAPRDHCAGLVTLPEAGGRGGVDYQLLTLVL
LAVNALLVLLALAAWGSRWLRRKLRARRKGGAPVHVRHMYSTRRPLRSMG
TGVSADFSGFQSHRPRTTVCALSEADLIEFPCDRFMDSTGGGTSGSLRRE
DHLLQRFAD

The amino sequence of the human Toll-like Receptor 4 (TLR4) protein has been previously defined and this shown below as SEQ ID NO:3.

MELNFYKIPDNLPFSTKNLDLSFNPLRHLGSYSFFSFPELQVLDLSRCEI
QTIEDGAYQSLSHLSTLILTGNPIQSLALGAFSGLSSLQKLVAVETNLAS
LENFPIGHLKTLKELNVAHNLIQSFKLPEYFSNLTNLEHLDLSSNKIQSI
YCTDLRVLHQMPLLNLSLDLSLNPMNFIQPGAFKEIRLHKLTLRNNFDSL
NVMKTCIQGLAGLEVHRLVLGEFRNEGNLEKFDKSALEGLCNLTIEEFRL
AYLDYYLDDIIDLFNCLTNVSSFSLVSVTIERVKDFSYNFGWQHLELVNC
KFGQFPTLKLKSLKRLTFTSNKGGNAFSEVDLPSLEFLDLSRNGLSFKGC
CSQSDFGTTSLKYLDLSFNGVITMSSNFLGLEQLEHLDFQHSNLKQMSEF
SVFLSLRNLIYLDISHTHTRVAFNGIFNGLSSLEVLKMAGNSFQENFLPD
IFTELRNLTFLDLSQCQLEQLSPTAFNSLSSLQVLNMSHNNFFSLDTFPY
KCLNSLQVLDYSLNHIMTSKKQELQHFPSSLAFLNLTQNDFACTCEHQSF
LQWIKDQRQLLVEVERMECATPSDKQGMPVLSLNITCQMNKTIIGVSVLS
VLVVSVVAVLVYKFYFHLMLLAGCIKYGRGENIYDAFVIYSSQDEDWVRN
ELVKNLEEGVPPFQLCLHYRDFIPGVAIAANIIHEGFHKSRKVIVVVSQH
FIQSRWCIFEYEIAQTWQFLSSRAGIIFIVLQKVEKTLLRQQVELYRLLS
RNTYLEWEDSVLGRHIFWRRLRKALLDGKSWNPEGTVGTGCNWQEATSI

Toll-like Receptor 14 and CD14 share a very low level of sequence homology, however, they exhibit structural homology. For example, CD14 exhibits a series of leucine-rich repeats, this series of leucine-rich repeats also being evident in the structure of TLR14. Further, CD14 has the same solenoid structure found in the ectodomain of Toll-like Receptors. CD14 differs from Toll-like Receptors in that it does not have a TIR signalling domain.

The amino acid sequence of human CD14 is provided below as SEQ ID NO:4.

MERASCLLLLLLPLVHVSATTPEPCELDDEDFRCVCNFSEPQPDWSEAFQCVSAVEVEIHA
GGLNLEPFLKRVDADADPRQYADTVKALRVRRLTVGAAQVPAQLLVGALRVLAYSRLKETL
EDLKITGTMPPLPLEATGLALSSLRLRNVSWATGRSWLAELQQWLKPGLKVLSIAQAHSPA
FSCEQVRAFPALTSLDLSDNPGLGERGLMAALCPHKFPAIQNLALRNTGMETPTGVCAALA
AAGVQPHSLDLSHNSLRATVNPSAPRCMWSSALNSLNLSFAGLEQVPKGLPAKLRVLDLSC
NRLNRAPQPDELPEVDNLTLDGNPFLVPGTALPHEGSMNSGVVPACARSTLSVGVSGTLVL
LQGARGFA

In certain embodiments of the present invention, it may be appropriate to substitute the human form of Toll-like Receptor 14 as defined in SEQ ID NO:1, with the murine form of Toll-like Receptor 14 as defined in SEQ ID NO:2.

Human TLR14, as defined in SEQ ID NO:1, contains 12 leucine rich repeats, a signal sequence and a putative transmembrane domain. Expression of human TLR14 is particularly evident in the brain, lung and ovary. The expression of human TLR14 is enhanced by microbial products, such as LPS (lipopolysaccaharide).

The inventors have identified that LPS stimulation results in a decrease in the association of TLR14 with CD14 and an increase of TLR14 interaction with TLR4. This observed interrelationship has been identified by the inventors as having utility in the assay methods for identifying modulators of Toll-like Receptor 4.

### Assays

The invention extends to assay methods and screening methods for determining modulators of the CD14/TLR14 interaction. As used herein, an “assay method” or “assay system” encompasses all the components required for performing and analysing the results of an assay that detects and/or measures a particular event or events. It is preferred, though not essential, that the screening assays employed in the present invention are high throughput or ultra high throughput and thus provide an automated, cost-effective means of screening.

### Antibodies and Related Binding Compounds

In certain embodiments, the invention extends to the use of antibodies and binding compounds derived therefrom or related thereto for the inhibition of the association of CD14 with Toll-like Receptor 14, or the association of CD14 with Toll-like Receptor 4. Further, said antibody or binding compound could have utility in preventing the binding of the binding of a Toll-like Receptor 4 agonist to Toll-like Receptor 4, CD14 or Toll-like Receptor 14.

An “antibody” is an immunoglobulin, whether naturally derived or partly or wholly synthetically produced. The term also covers any polypeptide, protein or peptide having a binding domain that is, or is homologous in function to, an antibody binding domain. Said polypeptides or proteins can be derived from natural sources, or they may be partly or wholly synthetically produced. Examples of antibodies are the immunoglobulin isotypes, for example IgG, IgA, IgM, IgE and the like as well as their isotypic subclasses, for example, IgG1, IgG2 and IgG3. The term further extends to antibody fragments which comprise an antigen binding domain and therefore exhibit binding specificity, such as Fab, F(ab′)2, scFv, Fv, dAb, Fd, fragments and bi-specific antibodies.

In various embodiments, the antibody for use in the invention may be a polyclonal antibody, a chimeric antibody, or a synthesized or synthetic antibody. In certain embodiments, the antibody may be a Camelid antibody, in particular a Camelid heavy chain antibody. Further, the antibody fragment may be a domain antibody or a nanobody derived from a Camelid heavy chain antibody. In certain embodiments the antibody may be a shark antibody or a shark derived antibody.

In certain embodiments, the antibody is an “isolated antibody”, this meaning that the antibody is (1) free of at least some proteins with which it would normally be found, (2) is essentially free of other proteins from the same source, e.g., from the same species, (3) is expressed by a cell from a different species, or (4) does not occur in nature.

As antibodies can be modified in a number of ways, as such the term “antibody” should be construed herein as covering any binding member or substance having a binding domain with the required specificity to Toll-like Receptor 14 or Toll-like Receptor 4. The antibody of the invention may be a monoclonal antibody, or a fragment, derivative, functional equivalent or homologue thereof. The term includes any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in European Patent Application Publication Number EP 0,120,694 and European Patent Application Publication Number EP 0,125,023.

The constant region of the antibody may be of any suitable immunoglobulin subtype, however it is preferred that the antibody subtype is IgG1. However, in alternative embodiments, the subtype of the antibody may be of the class IgA, IgM, IgD and IgE where a human immunoglobulin molecule is used. Such an antibody may further belong to any subclass e.g. IgG1, IgG2a, IgG2b, IgG3 and IgG4.

Fragments of a whole antibody can perform the function of antigen binding. Examples of such binding fragments are; a Fab fragment comprising of the VL, VH, CL and CH1 antibody domains; an Fv fragment consisting of the VL and VH domains of a single antibody; a F(ab′)2 fragments, a bivalent fragment comprising two linked Fab fragments; a single chain Fv molecule (scFv), wherein a VH domain and a VL domain are linked by a peptide linker which allows the two domains to associate to form an antigen binding site; or a bi-specific antibody, which may be multivalent or multispecific fragments constructed by gene fusion.

A fragment of an antibody or of a polypeptide for use in the present invention, for example, a fragment of a TLR14, CD14 or TLR4 specific antibody (the latter in the case of an antibody which inhibits TLR14, or CD14 biological function by binding to TLR4 at an epitope which prevents TLR14 or CD14 complexing with TLR4 as a co-receptor), generally means a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” of such an antibody or polypeptide, or of a fragment of a CD14, TLR14 or TLR4 specific antibody means an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having TLR14 and/or CD14 and/or TLR4 binding activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

### Production of Antibodies

The antibodies for use in the binding assays of the present invention may be provided by a number of techniques. For example, a combinatorial screening technique such as a phage display-based biopanning assay may be used in order to identify amino acid sequences which have binding specificity to binding epitopes present on TLR14, CD14 or TLR4. Such phage display biopanning techniques involve the use of phage display libraries, which are utilised in methods which identify suitable epitope binding ligands in a procedure which mimics immune selection, through the display of antibody binding fragments on the surface of filamentous bacteria. Phage with specific binding activity are selected. The selected phage can thereafter be used in the production of chimeric, CDR-grafted, humanised or human antibodies.

In certain embodiments, the antibody is a monoclonal antibody, which may be produced using any suitable method which produces antibody molecules by continuous cell lines in culture. Suitable methods will be well known to the person skilled in the art and include, for example, the method of Kohler and Milstein (Kohler et al. Nature, 256, 495-497. 1975). Chimeric antibodies or CDR-grafted antibodies with binding specificity to TLR14, CD14 or TLR4 are further provided within the scope of the present invention. In certain embodiments, the antibodies of the invention may be produced by the expression of recombinant DNA in host cell.

In certain embodiments, the monoclonal antibodies may be human antibodies, produced using transgenic animals, for example, transgenic mice, which have been genetically modified to delete or suppress the expression of endogenous murine immunoglobulin genes, with loci encoding for human heavy and light chains being expressed in preference, this resulting in the production of fully human antibodies.

In certain embodiments the antibodies may be humanized antibodies. Humanized antibodies may be produced, for example, by the method of Winter as described in U.S. Pat. No. 5,585,089. A humanised antibody may be a modified antibody having the hypervariable region of a monoclonal antibody such as a TLR14, CD14 or TLR4 specific antibody and the constant region of a human antibody. Thus the binding member may comprise a human constant region. The variable region other than the hypervariable region may also be derived from the variable region of a human antibody and/or may also be derived from a monoclonal antibody such as a TLR14, CD14 or TLR4 specific antibody. In such case, the entire variable region may be derived from the murine monoclonal antibody and the antibody is said to be chimerised. Methods for making chimeric antibodies are known in the art. Such methods include, for example, those described in U.S. Pat. Nos. 4,816,397 and 4,816,567, of Boss and Cabilly respectively.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, European Patent Application No 0,184,187, GB Patent Application No. 2,188,638A or European Patent Application No. 0,239,400. A hybridoma or other cell producing an antibody may be subject to genetic mutation or other changes, which may or may not alter the binding specificity of antibodies produced.

The antibodies or antibody fragments of and for use in the present invention may also be generated wholly or partly by chemical synthesis. The antibodies can be readily prepared according to well-established, standard liquid or, preferably, solid-phase peptide synthesis methods, general descriptions of which are broadly available and are well known by the person skilled in the art. Further, they may be prepared in solution, by the liquid phase method or by any combination of solid-phase, liquid phase and solution chemistry.

Another convenient way of producing antibodies or antibody fragments suitable for use in the present invention is to express nucleic acid encoding them, by use of nucleic acid in an expression system.

In certain embodiments, where the TLR14 or CD14 inhibitory compound is an antibody, the antibody is administered to a subject in a therapeutically effective amount. In certain embodiments, the therapeutically effective amount comprises the antibody in a range chosen from 1 μg/kg to 20 mg/kg, 1 g/kg to 10 mg/kg, 1 μg/kg to 1 mg/kg, 10 μg/kg to 1 mg/kg, 10 μg/kg to 100 pg/kg and 500 pg/kg to 1 mg/kg.

Nucleic acid for use in accordance with the present invention may comprise DNA or RNA and may be wholly or partially synthetic. In a preferred aspect, nucleic acid for use in the invention codes for antibodies or antibody fragments of the invention as defined above. The skilled person will be able to determine substitutions, deletions and/or additions to such nucleic acids which will still provide an antibody or antibody fragment of the present invention.

Nucleic acid sequences encoding antibodies or antibody fragments for use with the present invention can be readily prepared by the skilled person using the information and references contained herein and techniques known in the art, given the nucleic acid sequences and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding antibody fragments may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

The nucleic acid may be comprised as constructs in the form of a plasmid, vector, transcription or expression cassette which comprises at least one nucleic acid as described above. The construct may be comprised within a recombinant host cell which comprises one or more constructs as above. Expression may conveniently be achieved by culturing under appropriate conditions recombinant host cells containing the nucleic acid. Following production by expression the antibody or antibody fragments may be isolated and/or purified using any suitable technique, then used as appropriate.

Systems for cloning and expression of a polypeptide in a variety of different host cells are well known. Suitable host cells include bacteria, mammalian cells, yeast, insect and baculovirus systems. Mammalian cell lines available in the art for expression of a heterologous polypeptide include Chinese hamster ovary (CHO) cells, HeLa cells, baby hamster kidney cells, NS0 mouse myeloma cells. A common, preferred bacterial host is E. coli. The expression of antibodies and antibody fragments in prokaryotic cells such as E. coli is well established in the art. Expression in eukaryotic cells in culture is also available to those skilled in the art as an option for production of a binding member.

General techniques for the production of antibodies are well known to the person skilled in the field, with such methods being discussed in, for example, Kohler and Milstein (1975) Nature 256: 495-497; U.S. Pat. No. 4,376,110; Harlow and Lane, Antibodies: a Laboratory Manual, (1988) Cold Spring Harbor, the contents of which are incorporated herein by reference.

Techniques for the preparation of recombinant antibody molecules are described in the above references and also in, for example, EP 0,623,679 and EP 0,368,684, which are incorporated herein by reference.

In certain embodiments of the invention, recombinant nucleic acids comprising an insert coding for a heavy chain variable domain and/or for a light chain variable domain of antibodies are employed. By definition such nucleic acids comprise coding single stranded nucleic acids, double stranded nucleic acids consisting of said coding nucleic acids and of complementary nucleic acids thereto, or these complementary (single stranded) nucleic acids themselves.

Furthermore, nucleic acids encoding a heavy chain variable domain and/or a light chain variable domain of antibodies can be enzymatically or chemically synthesised nucleic acids having the authentic sequence coding for a naturally-occurring heavy chain variable domain and/or for the light chain variable domain, or a mutant thereof.

Recombinant DNA technology may be used to improve the antibodies of the invention. Thus, chimeric antibodies may be constructed in order to decrease the immunogenicity thereof in diagnostic or therapeutic applications. Moreover, immunogenicity within, for example, a transgenic organism such as a pig, may be minimised, by altering the antibodies by CDR grafting in a technique analogous to humanising antibodies as described hereinbefore.

In order to reduce immunogenicity within a recipient, the invention may employ recombinant nucleic acids comprising an insert coding for a heavy chain variable domain of an antibody fused to a human constant domain. Likewise the invention concerns recombinant DNAs comprising an insert coding for a light chain variable domain of an antibody fused to a human constant domain kappa or lambda region.

Antibodies may also be generated by mutagenesis of antibody genes to produce artificial repertoires of antibodies. This technique allows the preparation of antibody libraries. Antibody libraries are also available commercially. Hence, the present invention advantageously employs artificial repertoires of immunoglobulins, preferably artificial scFv repertoires, as an immunoglobulin source in order to identify binding molecules which have specificity for TLR14 or CD14.

### Antibody Selection Systems

Immunoglobulins which are able to bind to TLR14 or CD14 and inhibit TLR4 activation and signalling, and which accordingly may be used in the methods of the invention, can be identified using any technique known to the skilled person. Such immunoglobulins may be conveniently isolated from libraries comprising artificial repertoires of immunoglobulin polypeptides. A “repertoire” refers to a set of molecules generated by random, semi-random or directed variation of one or more template molecules, at the nucleic acid level, in order to provide a multiplicity of binding specificities. Methods for generating repertoires are well characterised in the art.

Any library selection system may be used in conjunction with the invention. Selection protocols for isolating desired members of large libraries are known in the art, as typified by phage display techniques. Such systems, in which diverse peptide sequences are displayed on the surface of filamentous bacteriophage, have proven useful for creating libraries of antibody fragments (and the nucleotide sequences that encode them) for the in-vitro selection and amplification of specific antibody fragments that bind a target antigen. The nucleotide sequences encoding the VH and VL regions are linked to gene fragments which encode leader signals that direct them to the periplasmic space of E. coli and as a result the resultant antibody fragments are displayed on the surface of the bacteriophage, typically as fusions to bacteriophage coat proteins (e.g. pill or pVIII). Alternatively, antibody fragments are displayed externally on lambda phage capsids (phage bodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straight forward.

Methods for the construction of bacteriophage antibody display libraries and lambda phage expression libraries are well known in the art (for example, McCafferty et al. (1990) Nature 348 552-554. One particularly advantageous approach has been the use of scFv phage-libraries (see for example Huston et al., 1988, Proc. Natl. Acad. Sci. USA).

An alternative to the use of phage or other cloned libraries is to use nucleic acid, preferably RNA, derived from the B cells of an animal which has been immunised with the selected target.

Isolation of V-region and C-region mRNA permits antibody fragments, such as Fab or Fv, to be expressed intracellularly. Briefly, RNA is isolated from the B cells of an immunised animal, for example from the spleen of an immunised mouse or the circulating B cells of a llama, and PCR primers used to amplify VH and VL cDNA selectively from the RNA pool. The VH and VL sequences thus obtained are joined to make scFv antibodies. PCR primer sequences may be based on published VH and VL sequences.

### Peptidomimetics

Peptide analogues, such as peptidomimetics or peptide mimetics are non-peptide compounds with properties representative of a template peptide. Such peptide analogues are typically developed using computerised molecular modelling. Peptidomimetics which are structurally similar to peptides which have affinity and binding specificity to TLR14 or CD14 may be used to mediate similar prophylactic and therapeutic effects to polypeptides and proteins which are determined to have such TLR14 or CD14 inhibitory function.

Peptidomimetics are typically structurally similar to a template peptide, but have one or more peptide linkages replaced by an alternative linkage, by methods which are well known in the art. For example, a peptide which has a binding specificity to a TLR14 or CD14 epitope may be modified such that it comprises amide bond replacement, incorporation of non peptide moieties, or backbone cyclisation. Suitably if cysteine is present the thiol of this residue is capped to prevent damage of the free sulphate group. A peptide may further be modified from the natural sequence to protect the peptides from protease attack.

Suitably a peptide used as a TLR14 or CD14 inhibitory compound in the present invention may be further modified using at least one of C and/or N-terminal capping, and/or cysteine residue capping. Furthermore, a peptide for use in the present invention may be capped at the N terminal residue with an acetyl group. Suitably, a peptide of and for use in the present invention may be capped at the C terminal with an amide group. Suitably, the thiol groups of cysteines are capped with acetamido methyl groups.

### Combinatorial Library

Combinatorial library technology (Schultz, J S (1996) Biotechnol. Prog. 12:729-743) provides an efficient way of testing a potentially vast number of different substances for their ability to modulate the activity of a polypeptide, in this case, the biological activity of TLR14, CD14 or TLR4. Prior to, or as well as being screened for, modulation of activity, test compounds may be screened for their ability to interact with the polypeptide, e.g. in a yeast two-hybrid system (which requires that both the polypeptide and the test substance can be expressed in yeast from encoding nucleic acid). This may be used as a coarse screen prior to testing a substance for actual ability to modulate activity of the polypeptide.

The amount of test substance or compound which may be added to an assay of the invention will normally be determined by trial and error depending upon the type of compound used. Typically, from about 0.01 to 100 nM concentrations of putative inhibitor compound may be used, for example from 0.1 to 10 nM. Greater concentrations may be used when a peptide is the test substance.

### Production of Inhibitory Polypeptides

In certain further aspects, the compound which inhibits the biological function of TLR14 or CD14 association with TLR4, or which prevents TLR14 dissociating with CD14 in response to a TLR4 agonist is a polypeptide. Expression, isolation and purification of suitable polypeptides may be accomplished by any suitable technique.

A method for producing polypeptides comprises culturing host cells transformed with a recombinant expression vector encoding a polypeptide under conditions that promote expression of the polypeptide, then recovering the expressed polypeptides from the culture. The person skilled in the art will recognise that the procedure for purifying the expressed polypeptides will vary according to such factors as the type of host cells employed, and whether the polypeptide is intracellular, membrane-bound or a soluble form that is secreted from the host cell.

Any suitable expression system may be employed. The vectors include a DNA encoding a polypeptide or fragment of the invention, operably linked to suitable transcriptional or translational regulatory nucleotide sequences, such as those derived from a mammalian, avian, microbial, viral, bacterial, or insect gene. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired (E. coli) host cells, and a selection gene by which transformants are identified, are generally incorporated into the expression vector.

In addition, a sequence encoding an appropriate signal peptide (native or heterologous) can be incorporated into expression vectors. A DNA sequence for a signal peptide (secretory leader) may be fused in frame to the nucleic acid sequence of the invention so that the DNA is initially transcribed, and the mRNA translated, into a fusion protein comprising the signal peptide. A signal peptide that is functional in the intended host cells promotes extracellular secretion of the polypeptide. The signal peptide is cleaved from the polypeptide during translation, but allows secretion of polypeptide from the cell.

Suitable host cells for expression of polypeptides include higher eukaryotic cells and yeast. Prokaryotic systems are also suitable. Mammalian cells, and in particular CHO cells are particularly preferred for use as host cells. Appropriate cloning and expression vectors for use with mammalian, prokaryotic, yeast, fungal and insect cellular hosts are described, for example, in Pouwels et al. Cloning Vectors: A Laboratory Manual, Elsevier, N.Y., (1986) (ISBN 0444904018).

### Small Molecules

In certain further embodiments, the compound which inhibits the association of TLR14 with CD14, the association of CD14 with TLR4, the association of a Toll-like Receptor 4 agonist with CD14, the translocation of a Toll-like Receptor 4 agonist between CD14 and Toll-like Receptor 14, or which prevents the dissociation of TLR14 and CD14 in the presence of a TLR4 agonist is a small molecule.

Non-peptide “small molecules” are often preferred for many in-vivo pharmaceutical uses. Accordingly, a mimetic or mimic of a compound which is identified according to any one of the assay methods of the present invention as inhibiting the association of TLR14 with CD14 or of TLR14 or CD14 with TLR4 or preventing the dissociation of TLR14 and CD14 in the presence of a TLR4 agonist is a small molecule which is designed for pharmaceutical uses. The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. peptides are not well suited as active agents for oral compositions as they tend to be quickly degraded by proteases in the alimentary canal. Mimetic design, synthesis and testing may be used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been determined, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can also be used in this modelling process.

In a variant of this approach, the three-dimensional structure of the ligand and its binding partner are modelled. This can be especially useful where the ligand and/or binding partner change conformation on binding, allowing the model to take account of the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in-vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in-vivo or clinical testing.

### Inhibitory Nucleic Acids Molecules

The invention extends to methods of inhibiting the association of TLR14 with CD14, or CD14 with TLR4, the association of a Toll-like Receptor 4 agonist with CD14, the translocation of a Toll-like Receptor 4 agonist between CD14 and Toll-like Receptor 14, or the prevention the dissociation of TLR14 and CD14 in the presence of a TLR4 agonist is a small molecule, by administering compounds or compositions which suppress the expression of the TLR14 gene product.

Suppression of expression of CD14 and/or TLR14 may be achieved using a number of techniques which will be well known to the person of ordinary skill in the art. For example, suppression may be mediated by an inhibitory nucleic acid selected from the group comprising, but not limited to: an anti-sense oligonucleotide, anti-sense DNA, anti-sense RNA, ribozyme, iRNA, miRNA, sRNA, shRNA.

As such, in certain further aspects, the present invention extends to a method for the treatment and/or prophylaxis of a TLR4-mediated disease condition by administering to a subject a therapeutically effective amount of an inhibitory nucleic acid which blocks the expression of CD14 and/or Toll-like Receptor 14.

As herein defined, the terms “blocks” and “blocking” when used in relation to CD14 or TLR14 gene expression means silencing the expression of at least one gene which results in the expression of the Toll-like Receptor 14 protein and/or the CD14 protein.

Gene silencing is the switching off of the expression of a gene by a mechanism other than genetic modification. Gene silencing can be mediated at the transcriptional level or at the post-transcriptional level. Transcriptional gene silencing can results in a gene being inaccessible to transcriptional machinery, and can be mediated, for example, by means of histone modifications. Post-transcriptional gene silencing results from the mRNA of a gene being destroyed, thus preventing an active gene product, such as a protein, in the present case the TLR14 protein and/or the CD14.

Accordingly, the invention further extends to the administration to a subject of an effective amount of an inhibitory nucleic acid molecule, such as an RNAi (RNA interference) agent, for example an interfering ribonucleic acid (such as sRNA or shRNA) or a transcription template thereof, such as a DNA encoding an shRNA to at least one cell type, tissue or organ present in the subject in order to block the expression of the TLR14 protein or the CD14 protein.

In certain further embodiments, the inhibitory nucleic acid molecule may be an antisense RNA molecule. Antisense causes suppression of gene expression and involves single stranded RNA fragments which physically bind to mRNA, this blocking mRNA translation.

Techniques for the preparation of appropriate nucleic acid for use as inhibiting nucleic acids are well known to the person skilled in the art and are discussed further hereinafter.

According to a further aspect of the invention there is provided the use of an inhibitory nucleic acid which blocks the expression of the Toll-like Receptor 14 protein and/or the CD14 protein in the preparation of a medicament for the treatment and/or prophylaxis of a TLR4 mediated disease or condition. In certain embodiments, the TLR4-mediated disease is an inflammatory condition, which can be sepsis.

As such, various aspects of the present invention provide for the use of inhibiting nucleic acids for the silencing of TLR14 gene expression and or CD14 gene expression.

Double-stranded RNA induces potent and specific gene silencing through a process referred to as RNA interference (RNAi) or post transcriptional gene silencing (PTGS). RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. RNAi has become the method of choice for loss-of-function investigations in numerous systems including mammalian cell lines. To specifically silence a gene in most mammalian cell lines, small interfering RNAs (siRNA) are used because large dsRNAs (>30 base pairs) trigger the interferon response and cause nonspecific gene silencing.

The RNAi agents employed in are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By “oligoribonucleotide”, it is meant a ribonucleic acid that does not exceed about 100 nucleotides (nt) in length, and typically does not exceed about 75 nucleotides in length, where the length in certain embodiments is less than about 70 nucleotides. As described herein, the length of the duplex structures for use in the present invention can typically ranges from about 15 to 30 base pairs, more preferably from about 15 to 29 base pairs.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

The term “expression” with respect to a nucleic acid or gene sequence refers to transcription of a gene and, as appropriate, translation of the resulting mRNA transcript to a protein. Thus, as will be clear from the context, expression of a protein coding sequence results from transcription and translation of the coding sequence.

“Inhibition of gene expression” refers to the absence (or observable decrease) in the level of protein and/or mRNA product from a target gene. “Specificity” refers to the ability to inhibit the target gene without manifest effects on other genes of the cell. Confirmation of inhibiting can be obtained through the use of techniques which are well known to the person skilled in the art such as: Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed.

Depending on the assay, quantitation of the amount of TLR14 gene expression allows one to determine a degree of inhibition which is greater than 10%, 33%, 50%, 90%, 95% or 99% as compared to a cell not treated according to the present invention. Lower doses of administered active agent and longer times after administration of active agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

### RNAi

Accordingly, as indicated above, one aspect of the present invention provides methods of employing RNAi to inhibit or suppress the expression of TLR14 or CD14 in a suitable cell type. By the term “inhibiting expression”, it is meant that the level of expression of the TLR14 gene or coding sequence is reduced or inhibited by at least about 2-fold, usually by at least about 5-fold, e.g., 10-fold, 15-fold, 20-fold, 50-fold, 100-fold or more, as compared to a control. In certain embodiments, the expression of the TLR14 or CD14 target gene is reduced to such an extent that expression of the target TLR14 or CD14 gene/coding sequence is effectively inhibited. In this regard, inhibiting expression of a target gene means inhibiting the transcription or translation of a coding sequence such as genomic DNA, mRNA etc., into a polypeptide product such as a protein, in the present case, TLR14, CD14 or TLR4.

In certain embodiments, instead of the RNAi agent being an interfering ribonucleic acid, such as an siRNA or shRNA as described above, the RNAi agent may encode an interfering ribonucleic acid, for example an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, such as a plasmid vector, a viral vector.

Administration of the RNAi agent to the TLR14 or CD14 expressing cell may be effected by means of a viral vector, or by other protocols which will be known to the person of ordinary skill in the art. For example, the nucleic acids may be introduced into the cell by way of microinjection, or by the fusion of vesicles. For example, the RNAi agent can be directly injected into the target cell. The agent may be introduced in an amount which allows delivery of at least one copy per cell. Higher doses (e.g., at least 5, 10, 100, 500 or 1000 copies per cell) of the agent may yield more effective inhibition; lower doses may also be useful for specific applications.

### Antisense

Also provided by the present invention are antisense nucleic acids for use in the silencing of the expression of TLR14 and/or CD14 so that they cannot associate with each other and further mediate LPS trafficking in order to activate TLR4. The antisense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted TLR14 or CD14 gene, and inhibits expression of the targeted TLR14 or CD14 gene product.

Antisense molecules inhibit gene expression through various mechanisms, for example by reducing the amount of mRNA available for translation, through activation of RNAse H, or steric hindrance. One or a combination of antisense molecules may be administered, where a combination may comprise multiple different sequences. Antisense molecules may be produced by expression of all or a part of the target TLR14 or CD14 gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 16 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

Antisense oligonucleotides may be chemically synthesized by methods known in the art (see Wagner et al. (1993), supra, and Milligan et al., supra). Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

### Soluble Proteins

In certain further embodiments the compounds which inhibit Toll-like Receptor 14 or CD14 biological function are soluble proteins, such as a soluble form of TLR4, TLR14 or CD14.

Soluble polypeptides are capable of being secreted from the cells in which they are expressed. In general, soluble polypeptides may be identified (and distinguished from non-soluble membrane-bound counterparts) by separating intact cells which express the desired polypeptide from the culture medium, e.g., by centrifugation, and assaying the medium (supernatant) for the presence of the desired polypeptide. The presence of polypeptide in the medium indicates that the polypeptide was secreted from the cells and thus is a soluble form of the protein.

In certain embodiments, the soluble form of TLR4 may be provided as a fusion protein. In certain embodiments, said fusion protein is comprised of a soluble portion of the TLR4 receptor, typically the extracellular domain or a portion thereof, for example having the amino acid sequence of SEQ ID NO:3, conjoined to a secondary peptide. In certain embodiments, the secondary peptide is derived from an immunoglobulin, and is typically the Fc receptor binding protein derived from the heavy chain of an immunoglobulin, typically a human immunoglobulin. The inclusion of the Fc domain in the fusion protein prolongs the circulatory half-life of the therapeutic protein.

The soluble TLR14 amino acid sequence and the immunoglobulin Fc receptor binding portion may be joined by any suitable technique, but are typically linked by a covalent bond. However a non-covalent bond may also be used. Alternatively, the polypeptide sequences could be directly conjoined or could be joined by means of a linkage moiety or spacer. A linker moiety such as a hinge region derived from an immunoglobulin may be used. The hinge region serves not only to link the amino acid defining the antigenic polypeptide with the amino acid defining the FcR binding polypeptide of the immunoconjugate, but also provides increased flexibility of the immunoconjugate which can confer improved binding specificity. Typically, the linker acts primarily as a spacer. Typically the linker is comprised of amino acids linked together by peptide bonds. The linker may, for example, comprise from 1 to 20 amino acids. Suitably the linker may comprise amino acid residues which are sterically unhindered, such as glycine and alanine. Suitable forms of linker moieties, are described hereinafter.

The amino acid defining the antigenic fragment of the immunoconjugate may be linked to the linker moiety at either its N-(amino) or C-(carboxyl). Suitable conjugation and linkage techniques would be well known to those skilled in the art and may include, for example, conjugation by thio-ester crosslinking utilising cysteine residues of the Fc polypeptide. Alternatively, the conjugation can involve the use of chemical crosslinking molecules, such as the use of heterobifunctional crosslinking agents, such as succinimidyl esters, for example, 3-(2-pyridyldithio)propionate or succinimidyl acetylthioacetate (Molecular Probes Inc. Handbook, Chapter 5, section 5.3).

Further techniques which may have utility in the conjugation of the antigenic fragment to the Fc binding polypeptide would include the techniques described in published International Patent Applications No WO 94/04690 and WO 96/27011.

Conjugation may further be achieved by genetic means through the use of recombinant DNA techniques that are well know in the art, such as those set forth in the teachings of Sambrook et al. Molecular Cloning: A Laboratory Manual, 2 ed. Vol. 1, pp. 1.101-104, Cold Spring Harbor Laboratory Press, (1989) and F. M. Ausubel et al. Current Protocols in Molecular Biology, Eds. J. Wiley Press (2006), the relevant portions of which are incorporated herein by reference.

### Combination Medicaments

As described hereinbefore, in certain aspects, the present invention extends to combinational therapies wherein compositions or methods relate to the administration of compounds which inhibit the biological functional activity of CD14 and/or TLR14, and which are administered in combination with at least one further therapeutic compound which serves to suppress CD14 activity.

Typically the primary and secondary therapeutic compositions are given contemporaneously. In certain embodiments, the primary therapeutic composition (i.e. the compound which antagonises the functional activity of TLR14) and the secondary therapeutic compounds are administered simultaneously. In certain further embodiments, they are administered sequentially.

In certain embodiments, the combination therapy may comprise a TLR14 and/or CD14 functional inhibitor, such as an antibody, a peptide, a small molecule or a peptidomimetic, which is co-administered to a subject along with at least one of: a CD14 inhibitor, a cytokine inhibitor (such as, but not limited to an inhibitor of IL-1, IL-6, IL-8 and IL-15), and inhibitor of tumour necrosis factor, a growth factor inhibitor, an immunosuppressor, an anti-inflammatory, an enzymatic inhibitor, a metabolic inhibitor, a cytotoxic agent or a cytostatic agent.

A person of relevant skill in the field will recognise that the administration to a subject of a combination therapy can be advantageous in that it permits administration of a lower dose of therapeutic to a subject in order to achieve and associated therapeutically effective effect. The administration of a lower combined dose also results in the subject being exposed to a lower toxicity level derived from the administered compound. Furthermore, as the secondary therapeutic compounds which are administered as part of the combination therapy provided by the invention target different pathways, there is likely to be a synergistic improvement in the overall efficacy of the therapy. An improvement in efficacy would again result in the need for a lower dose to be administered and as such an associated reduction in toxicity.

Secondary compounds for use in suppressing the biological functional activity of CD14 and/or TLR14 may include, but are not limited to; soluble forms of CD14 or TLR14, peptide inhibitor compounds, peptidomimetics, small molecule, fusion proteins or ligands, and antibodies or antibody fragments.

### Pharmaceutical Compositions

The present invention extends to a pharmaceutical composition comprising a compound which inhibits the expression or biological functional activity of TLR14 or CD14. Pharmaceutical compositions according to and for use in accordance with the present invention may comprise, in addition to active ingredient (i.e. an inhibitor of TLR14 or CD14 expression or biological activity), a pharmaceutically acceptable excipient, carrier, buffer stabiliser or other materials well known to those skilled in the art. Examples of suitable pharmaceutical carriers include; water, glycerol, ethanol and the like.

The monoclonal antibody or fusion protein of the present invention may be administered to a patient in need of treatment via any suitable route. As detailed herein, it is preferred that the composition is administered parenterally by injection or infusion. Examples of preferred routes for parenteral administration include, but are not limited to; intravenous, intracardial, intraarterial, intraperitoneal, intramuscular, intracavity, subcutaneous, transmucosal, inhalation or transdermal.

Routes of administration may further include topical and enteral, for example, mucosal (including pulmonary), oral, nasal, rectal.

The formulation may be a liquid, for example, a physiologic salt solution containing non-phosphate buffer at pH 6.8-7.6, or a lyophilised or freeze dried powder.

In certain embodiments, the composition is deliverable as an injectable composition. For intravenous, intradermal or subcutaneous application, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection or, Lactated Ringer's injection. Preservatives, stabilisers, buffers, antioxidants and/or other additives may be included, as required.

The composition may also be administered via microspheres, liposomes, other microparticulate delivery systems or sustained release formulations placed in certain tissues including blood.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition ISBN 0-912734-04-3 and Pharmaceutical Dosage Forms and Drug Delivery Systems; Ansel, N. C. et al. 7th Edition ISBN 0-683305-72-7, the entire disclosures of which is herein incorporated by reference.

Dosage regimens can include a single administration of the composition of the invention, or multiple administrative doses of the composition. The compositions can further be administered sequentially or separately with other therapeutics and medicaments which are used for the treatment of the condition for which the fusion protein of the present invention is being administered to treat.

The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

### DEFINITIONS

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers. As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like.

The nomenclature used to describe the polypeptide constituents of the fusion protein of the present invention follows the conventional practice wherein the amino group (N) is presented to the left and the carboxy group to the right of each amino acid residue.

The expression “amino acid” as used herein is intended to include both natural and synthetic amino acids, and both D and L amino acids. A synthetic amino acid also encompasses chemically modified amino acids, including, but not limited to salts, and amino acid derivatives such as amides. Amino acids present within the polypeptides of the present invention can be modified by methylation, amidation, acetylation or substitution with other chemical groups which can change the circulating half life without adversely affecting their biological activity.

The terms “peptide”, “polypeptide” and “protein” are used herein interchangeably to describe a series of at least two amino acids covalently linked by peptide bonds or modified peptide bonds such as isosteres. No limitation is placed on the maximum number of amino acids which may comprise a peptide or protein. Furthermore, the term polypeptide extends to fragments, analogues and derivatives of a peptide, wherein said fragment, analogue or derivative retains the same biological functional activity as the peptide from which the fragment, derivative or analogue is derived

As used herein, the term “therapeutically effective amount” means the amount of an agent, binding compound, small molecule, fusion protein or peptidomimetic of the invention which is required to suppress a TLR4-mediated inflammatory condition.

As used herein, the term “prophylactically effective amount” relates to the amount of a composition which is required to prevent the initial onset, progression or recurrence of TLR4-mediated inflammatory condition, such as sepsis.

As used herein, the term “treatment” and associated terms such as “treat” and “treating” means the reduction of the progression, severity and/or duration of a TLR4 or TLR14 mediated condition or at least one symptom thereof, wherein said reduction or amelioration results from the administration of a compound which disrupts or prevents the association of TLR14 as a co-receptor with TLR4.

The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition.

As used herein, the term “subject” refers to an animal, preferably a mammal and in particular a human. In a particular embodiment, the subject is a mammal, in particular a human. The term “subject” is interchangeable with the term “patient” as used herein.

As herein defined, the term “association” when used in relation to Toll-like Receptor 14 and CD14 means that Toll-like Receptor 14 and CD14 interact in a manner which results in them complexing. As such, the term “association” means that the Toll-like Receptor 14 and the CD14 interact, associate or complex. This association, interaction or complexing may be transient. That is, the association, interaction or complexing may end following a particular intracellular event.

### EXAMPLES

The present invention will now be described with reference to the following examples which are provided for the purpose of illustration and are not intended to be construed as being limiting on the present invention.

### Example 1

### Binding of Toll-Like Receptor 14 to Toll-Like Receptor 2 and Toll-like Receptor 4

This experiment was designed to identify whether Toll-like Receptor 14 would bind to Toll-like Receptor 2 or Toll-like Receptor 4, each of which were expressed on transfected HEK293 (human embryonic kidney) cells.

### Material and Methods:

HEK293 cells were seeded at 2×10⁵ cells/ml. After 24 hours Flag tagged TLR2 and Flag tagged TLR4 were transfected into the HEK293 cells using GENEJUICE transfection reagent (Novagen) according to manufacturer's instructions. After 24 hours, the TLR4 transfected HEK293 cells were either left untreated or treated with 100 ng of the TLR4 agonist LPS (lipopolysaccharide) for 2 hours. At the same time point (24 hours) the TLR2HEK293 transfected cells were either left untreated or treated with 1 mg/ml of the TLR2 agonist Pam2Cys4 for 2 hours.

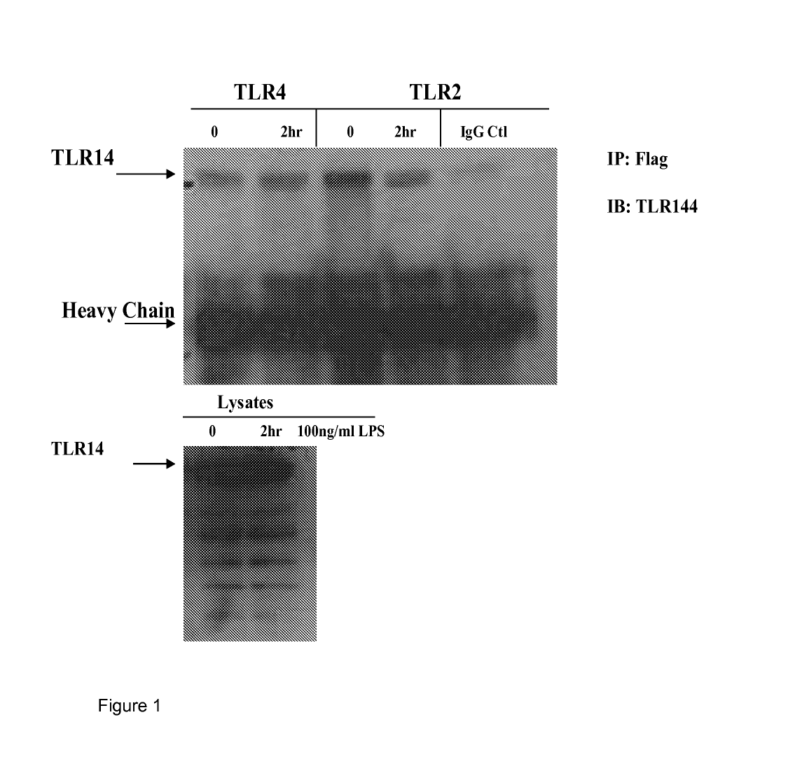

The cells were then lysed in low stringency lysis buffer and incubated with flag beads. Samples were resolved on 10% SDS gels and immunoblotted for TLR14, this being indicative of TLR14 binding. The results are shown by a series of gels shown in FIG. 1.

### Results:

FIG. 1 shows that endogenous TLR14 binds to HEK293 cells which have been transfected with either TLR4 or TLR2, both of which are over-expressed in the transfected HEK293 cells. Binding between TLR14 and TLR2 is observed following stimulation of TLR2 with the TLR2 agonist Pam2Cys4. Binding between TLR4 and TLR14 is also observed following stimulation of TLR4 with the TLR4 agonist LPS. No binding is seen with the immunoglobulin control (IgG Ctl). TLR14 is further identified in cell lysates after 2 hours of stimulation of TLR4 transfected cells with LPS.

### Example 2

### Effect of LPS Stimulation on Binding of TLR14 to TLR4

This experiment was designed to determine the effect of LPS on the binding of TLR14 to TLR4 transfected HEK293 cells.

### Materials and Methods:

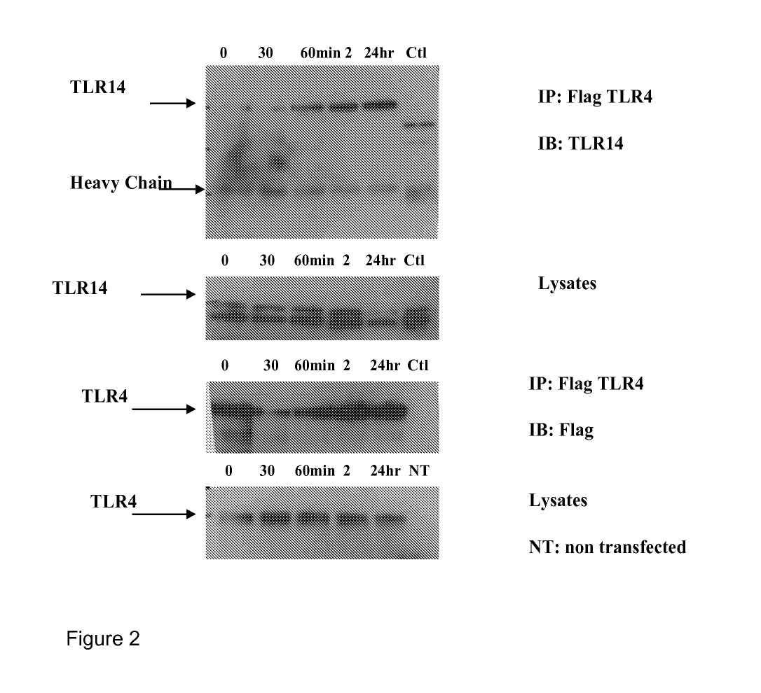

HEK293 cells were seeded at 2×10⁵ cells/ml. After 24 hours, Flag tagged TLR4 was transfected into the HEK293 cells using GENEJUICE transfection reagent (Novagen) according to the manufacturer's instructions. After an additional 24 hours, cells were stimulated with 100 ng/ml of the TLR4 agonist LPS (lipopolysaccharide) for 0 minutes, 30 minutes, 60 minutes, 2 hours, or 24 hours. Cells were lysed in low stringency lysis buffer, and then incubated with Flag beads. Samples were resolved on 10% SDS gels and immunoblotted to determine the presence of TLR14. The results are shown in FIG. 2.

### Results:

FIG. 2 shows a series of gels illustrating that upon incubation of TLR4 transfected HEK293 cells with LPS, the association between TLR14 and TLR4 increases over time, the strongest effect being evident at the 24 hour time point. The observed results therefore constitute a signal for LPS.

### Example 3

### Characterisation of Association of Toll-like Receptor 14 to CD14

In this experiment the effect of the addition of LPS on the association of TLR14 with CD14 is assessed.

### Materials and Methods:

HEK293 (human embryonic kidney) cells were seeded at 2×10⁵ cells/ml. After 24 hours, CD14 was transfected into the HEK293 cells using GENEJUICE transfection reagent (Novagen) according to the manufacturer's instructions.

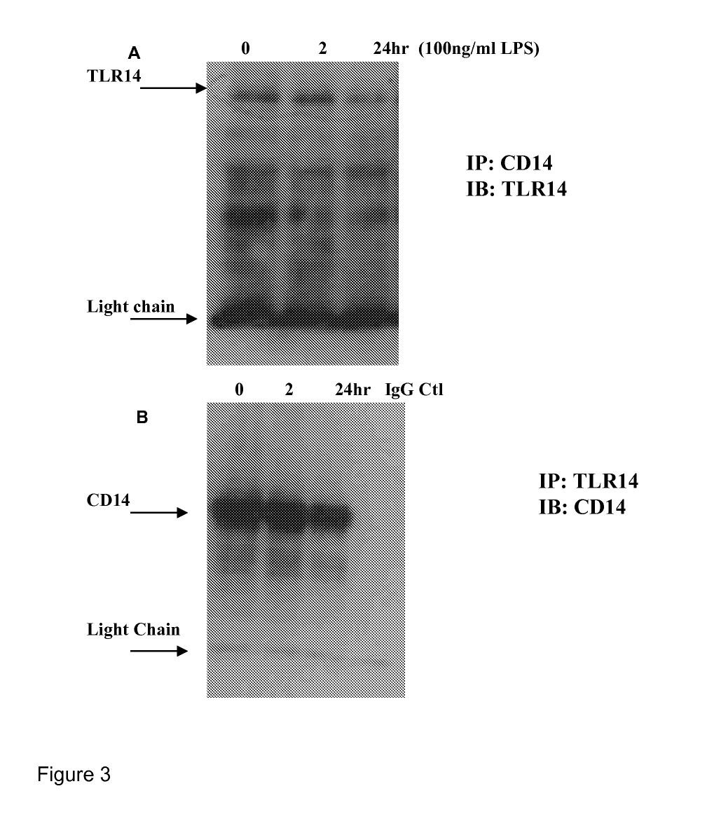

After 24 hours had elapsed, cells were stimulated with 100 ng/ml LPS (lipopolysaccharide) for 0 hours, 2 hours or 24 hours. After 2 and 24 hours, cells were lysed in low stringency lysis buffer, then incubated in protein A/G beads pre-coupled with an antibody which has binding specificity for CD14 (FIG. 3A) or with protein A/G beads precoupled with an antibody which has binding specificity for CD14 (FIG. 3B). Samples were resolved on 10% SDS gels and immunoblotted for TLR14 (FIG. 3A) and CD14 (FIG. 3B) respectively. An IgG immunoglobulin control (IgG Ctl) is shown in FIG. 3B.

### Results:

FIGS. 3A and 3B show that TLR14 expressed by HEK293 cells can interact with over-expressed CD14. This interaction decreases upon treatment of the cells with LPS. This decrease in the interaction of TLR14 and CD14 is observed following an incubation time of 2 hours, but is far more markedly observed following an incubation time of 24 hours.

### Example 4

### Determination of Inter-Relationship of TLR14 and CD14 to LPS-Mediated TLR4 Activation and Signalling

In order to further determine the specific role of CD14 and TLR14 in LPS-mediated TLR4 activation and signalling, a number of experiments were performed where the presence of either or both of CD14 and/or TLR14 was varied. These experiments were designed to allow an assessment to be made as to whether both CD14 and TLR14 must be present in order to allow activation and signalling of TLR4 following the exposure of TLR4 to the agonist LPS.

### Materials and Methods

### (i) Cell Culture—Growth and Maintenance of Cell Lines

All cell lines used were stored in liquid nitrogen. All cells were stored at a concentration of 1×10⁷ cells/ml in 95% foetal calf serum (FCS) and 5% dimethyl sulphate (DMSO) in plastic cryogenic vials. Cells were thawed at 37° C., 1 ml of FCS was added to the cells and then they were placed in the media specific to each cell line.

U373, HEK293, HEK293T and HEK293TLR4 (TLR4 transfected HEK 293) cells were placed in 10 ml of DMEM medium (Gibco) (10% FCS), THP1 cells were placed in 10 ml of RPMI 1640 medium (Gibco) (10% FCS). Cells were centrifuged at 1000×g for 3 minutes. The pellet of cells were then resuspended in 10 ml of complete medium specific to the cell line, which was then placed in a T25 cell culture flask. Cells were maintained at 37° C. with 5% CO₂. In order to use the cells in experimental assays they were grown in T175 cell culture flasks. For use in transfection assays, HEK293, HEK293TLR4, MEF (mouse embryonic fibroblast cells) and U373 cells were typically seeded at 1×10⁵ cells/ml in 96 well, 6 well or 10 cm dishes 24 hours prior to transfection, whereas THP1 cells were seeded at 2×10⁵ cells/ml 24 hours prior to transfection. For continuing cell culture, all cell lines were seeded at 1×10⁵ cells/ml and sub-cultured two or thee times a week. HEK293, U373 and MEF cells were removed from the surface of the cell culture flask by initially washing cells with 2 ml of TRYPSIN-EDTA followed by incubation with 5 ml of TRYPSIN-EDTA for 5 minutes. 10 ml of complete media was added to the cells and they were centrifuged at 1000×g for 5 minutes. The contents of the flask were then transferred into a 30 ml Sterilin container and centrifuged at 1000×g for 5 minutes. THP1 cells are a suspension cell line and therefore the cells can be poured directly into a sterile falcon tube and centrifuged at 1000×g for 5 minutes. In all cases, the supernatant was removed from the cells and the pellet of cells was resuspended in 1 ml of complete media. Cells were counted using a haemocytometer and light microscope. Cell viability was determined using the dye Trypan blue, which is excluded from healthy cells but taken up by non-viable cells.

Blood was obtained either directly from a healthy donor or taken from the blood bank at St. James' hospital (Dublin, Ireland). Blood was transferred into a 50 ml falcon tube and diluted 1:2 with phosphate buffered saline (PBS). Ficoll-Pague PLUS (Amersham) was used to separate the blood into red blood cells, white blood cell ring and serum. The blood was slowly added to 20 ml of Ficoll-Pague PLUS. The tubes were centrifuged at 1700×g for 30 minutes. The white blood cell ring was transferred into a new 50 ml tube using a Pasteur pipette. The volume was adjusted to 50 ml and the samples were centrifuged again at 1700×g for 10 minutes. The supernatant was removed. This step was repeated again, the pellet was then resuspended in 10 ml of complete IMDM media (10% FCS, 0.1 Ciprofloxacin (10 mg/ml). Cells were counted and seeded at a concentration of 2×10⁶ cells/ml. For 24 well plates 500 μl of media was added to each well, and for 6 well plates 3 ml of media was added to each well.

### RNA Isolation

RNA isolations were carried out on the U373/CD14, THP1 and MEF cell lines and also on the human PBMC. 1×10⁷ cells were obtained for the RNA isolation procedure. The cells were seeded in 10 cm dishes. The media was then removed; the cells were washed gently with 2 ml of sterile PBS. RNA is extracted using the RNeasy minikit (Qiagen). Cells were harvested in two different ways depending upon whether the cells grew in suspension (THP1 cells) or in a monolayer (U373/CD14 cells and the like).

Cells that grew in suspension were counted as described above. 1×10⁷ cells/ml were centrifuged at 300×g in a centrifuge tube for 5 minutes. All supernatant was aspirated.

In the case of cells which grew in a monolayer, all media on the cells were aspirated off and cells were lysed directly in the 10 cm dish as described below.

For pelleted cells, the pellet was loosened thoroughly by flicking the tube. 600 μl of buffer RLT was added to the cells. In order to lyse cells directly in the 10 cm dish, 600 μl of buffer RLT was added to the dish. The monolayer of cells was disrupted using a cell scraper. The lysate was collected into a microcentrifuge tube and vortexed for 1 minute.

### Homogenisation of the Lysate

The lysate was passed up to 5 times though a blunt 20 gauge needle (0.9 mm in diameter) fitted to an RNase-free syringe. 1 volume of 700 μl ethanol was added to the homogenised lysate and mixed well by pipetting. 700 μl of the sample was transferred to an RNeasy spin column in a 2 ml collection tube. The samples were centrifuged at 8000×g for 15 seconds. The flow through was discarded. 700 μl of buffer RW1 was added to the sample and again it was centrifuged at 8000×g for 15 seconds and the flow through was discarded. 500 μl of buffer RPE was added to the column and it was centrifuged at 8000×g for 15 seconds and the flow through was discarded. 500 μl of buffer RPE was added to the column and it was centrifuged at 8000×g for 2 minutes and the flow though was discarded. The column was centrifuged for an additional 2 minutes and the flow through was discarded. The RNeasy spin column was placed in a sterile Eppendorf tube and 30 μl of RNase free water was added. The column was centrifuged at 8000×g for 1 minute in order to elute the RNA off the column. RNA was quantified using a spectrophotometer by reading at an optical density (OD) of 260 (DNA), and 280 (proteins). Ratios of 260/280 are expected to be approximately 1.8/2 for pure RNA.

### (ii) Reverse Transcription Polymerase Chain Reaction (RT-PCR)

Production of cDNA from the RNA Template

Following RNA quantification, the concentration of each sample was normalised by diluting more concentrated samples with Rnase free water. Once the concentration was equal in each sample for each template, 4 μl of RNA and 1 μl of random primers were added to a minifuge tube. The sample was heated to 70° C. for 5 minutes and 4° C. for 5 minutes. This process produced denatured RNA. The next step was reverse transcription. Table 1 lists the components required. These were added to a minifuge tube prior to the addition of the template and primers.

TABLE 1

| | Experimental reaction | Water control |
|---|---|---|
| | (μl) | (μl) |
| Nuclease free water | 5.5 | 5.5 |
| Improm-II 5 × Reaction | 4.0 | 4.0 |
| buffer | | |
| MgCl₂, 25 mM | 3.0 | 3.0 |
| dNTPs, 10 mM | 1.0 | 1.0 |
| RRNasin | 0.5 | 0.5 |
| Improm-II reverse | 1.0 | 1.0 |
| transcription (RT) | | |
| Total volume | 15.0 | 15.0 |

Each component was added into minifuge tubes in the order depicted on the table above. All samples were kept on ice throughout the experiment. 5 μl of denatured RNA was added to each minifuge tube. The tubes were placed into the PCR machine and the following program was used: (i) 25° C. for 5 minutes (annealing occurs), (ii) 42° C. for 60 minutes (initial cDNA strand synthesis), (iii) 70° C. for 15 minutes (activation of reverse transcription). Once the cDNA was synthesized it could be stored at this stage at 4° C., or used directly in a PCR reaction.

### Polymerase Chain Reaction (PCR)

Each component required is outlined in Table 2, which is shown below. In order to save time, master mixes for each PCR reaction were made up in sterile Eppendorf tubes and kept on ice. Filter tips were used for all mixes made up and used in the PCR reactions.

A total of 20 μl of the master mix was needed for each PCR reaction. A total of 8 reactions were carried out for each experiment (in order to allow for pipetting error, mix was made up for 9 samples). Table 2 shows the components needed for the master mixes.

TABLE 2

| | Experimental | Master Mix μl | Water control μl |
|---|---|---|---|
| | reaction | (9 samples) | (9 samples) |
| 10 × Reaction | 2.5 | 22.5 | 22.5 |
| Buffer | | | |
| dNTP mix, 10 mM | 0.5 | 4.5 | 4.5 |
| MgCl₂, 2.25 mM | 0.75 | 10.125 | 10.125 |
| Template | 1 | — | 5 |
| Taq DNA | 0.125 | 1.125 | 1.125 |
| polymerase | | | |
| Nuclease free | 14.125 | 141.75 | 136.75 |
| water | | | |
| Total volume | 20.0 | 180.0 | 180.0 |

### RT-PCR Primers

| Forward KIAA0644 (TLR14) primer: | |
|---|---|
| GCCTTGCGCCTCCTGCTCGTGGTG | (SEQ ID NO: 5) |
| Reverse KIAA0644 (TLR14) primer: | |
| CCACCGCGAGAGCTTCTCGAAGGT | (SEQ ID NO: 6) |
| (310bp) | |
| Forward GapDH primer: | |
| GAACGGGAAGCTTGTCATCAA | (SEQ ID NO: 7) |
| Reverse GapDH primer: | |
| CTAAGCAGTTGGTGGTGCAG | (SEQ ID NO: 8) |
| (350bp) | |

Primers were designed and obtained (Eurofins MWG Operon, Alabama, USA). The primers were made up at a concentration of 100 pM (pico molar). They were then made up to working stocks of 10 pM. Each primer was made up into a mix consisting of the forward and reverse primers (10 μl of the forward primer and 10 μl of the reverse primer and 180 μl water (molecular biology grade water)). 5 μl of each of the primer mixes was used per reaction, bringing the total volume in each PCR tube to 25 μl. When all the samples were prepared in sterile PCR tubes they were placed into a preheated PCR machine (preheated to 95° C.).

The following PCR program was used: 5 cycles of the following: (i) 95° C. for 5 minutes (denaturation), (ii) 97° C. for 20 seconds, (iii) 64° C. for 1 minutes (Annealing), (iv) 72° C. for 45 seconds (Extension). 35 cycles of the following was also used: (i) 96° C. for 20 seconds, (ii) 62° C. for 45 seconds, (iii) 72° C. for 1 minute. The mixture was then held at 72° C. for 7 minutes. Then a 4° C. hold followed.

The annealing temperature had to be varied in different experiments as it is based on the melting temperature of the primers. Therefore each PCR reaction had to be optimized for each primer pair.

### Agarose Gel Electrophoresis

In order to visualise the PCR products, a 1% agarose gel was made up using the following: 150 ml of TAE (tris-Acetate-EDTA), 1.5 g of agarose, and 5 μl of Ethidium bromide (EtBr).

When the gel set, the combs were removed and the gel was placed into a gel box filled with TAE running buffer. 2 μl of DNA loading buffer (50% (v/v) sterile glycerol in sterile H₂0), and 10 mg of bromophenol blue was added to 20 μl of the PCR product. 15 μl of this was loaded onto the gel. A molecular weight marker was made up by adding 5 μl of a 1 KB marker and 1 μl of loading buffer. The gel was connected to a power supply and it was maintained at 100V for approximately 2 hours. The gel was then visualized using a UV gel docking system.

(iii) Transient Transfection Using GeneJuice

GENEJUICE™, the liposomal based transfection reagent from Novagen was used to transfect HEK293 cells, MEF cells and A172 cells (2×10⁵ cells/ml) in 96 well plates. Cells were transfected with different concentrations (10 ng, 20 ng, 50 ng, 100 ng) of promoters in PGL3 basic and also in the PGL3 enhancer vectors. In all cases the amount of DNA used per transfection was normalised using the appropriate amount of relevant empty vector control. Control plasmids used were the kB reporter plasmid and the normalization control plasmid, TK renilla. The kB reporter plasmid is used to test to ensure the cells are responding to certain Toll-like Receptor ligand agonists, such as LPS (TLR4) and Pamcys (TLR2). 0.8 μl of GENEJUICE™ was mixed with 9.2 μl of serum free DMEM per transfection and incubated at room temperature for 5 minutes. 30 μl of this was then added to DNA and incubated for 15 minutes at room temperature. Each DNA transfection was carried out in triplicate. 10 μl of the DNA and GENEJUICE™ mix was added to the cells, which were incubated at 37° C. for 16 hours prior to stimulation. For 6 well plate transfections, the total DNA used was 1-2 μg, 8 μl GENEJUICE™ and 92 μl serum-free DMEM. To transfect 10 cm dish, 5-10 μg of DNA was used in combination with 15 μl GENEJUICE™ and 235 μl serum-free DMEM.

### (iv) Western Blot Analysis

### Cell Stimulation and Extract Preparation

Cells were seeded at 2×10⁵ cells/ml in 6 cm dishes and stimulated with a number of TLR ligands (LPS, Polyl:C, Malp 2 and Pam3Cys4) for a number of different time points. The reactions were terminated by removal of media from the cells followed by the addition of PBS to the dishes. The cells were washed for a total of 3 times in PBS. Cells were either lysed directly using sample buffer or they were lysed in high stringency buffer or RIPA buffer.

Cell Lysis with Sample Buffer

100 μl of sample buffer (containing 10% mercaptoethanol) was added to each well of a 6 well plate. Cells were removed using a rubber cell scraper and transferred into Eppendorf tubes. The samples were then sonicated for 10 seconds at 80% strength, boiled at 100° C. for 5 minutes and then centrifuged at 17,900×g for 5 minutes.

### Sodium Dodecyl Sulfate-polyacrylamide Gel Electrophoresis (SDS-PAGE)

Samples were resolved on Sodium Dodecylsulphate (SDS) polyacrylamide gel using a constant current of 25 mA per gel. Samples were first electrophoresed though a stacking gel (1 ml 30% bisacrylamide mix, 0.75 ml 1M Tris pH 6.8, 60 μl 10% ammonium persulphate and 6 μl TEMED made up to 6 ml with H₂O) to condense protein, and then resolved according to size using 8-12% polyacrylamide gels (30% bisacrylamide mix, 3.75 ml 1.5M tris pH 8.8, 150 μl 10% (w/v) ammonium persulphate, 6 μl TEMED made up to 15 ml with H₂O). Pre-stained protein markers (New England Biolabs) were also placed on the gel as molecular weight standards.

### (v) Transfer of Proteins to Membrane

The resolved proteins were then transferred to polyvinylidene diflouride (PVDF) using a wet transfer system with all components soaked first in transfer buffer (25 mM Tris-HCL pH 8.0, 0.2 M glycine, 20% methanol. The gel was placed on a layer of filter paper and sponge overlaid with the membrane. A second piece of filter paper was placed on top followed by a second sponge. The entire assembly was placed in a cassette. An ice pack was placed in the chamber, which was then filled with transfer buffer and a constant current of 150 mA was applied for 2 hours.

### (vi) Blocking the Membrane

Membranes were blocked for non specific binding by incubation in 50 ml of 5% (w/v) non fat dried milk in 1% (v/v) Tris Buffered Saline (TBS)-Tween for 1 hour at room temperature. The membrane was washed three times for 5 minutes in 1% (v/v) TBS-Tween.

(vii) Antibody Incubation

The membrane was incubated for 1 hour at room temperature or overnight at 4° C. with the primary antibody of interest at 1:100 to 1:1000 dilution depending on the particular antibody. Following incubation the membrane was washed for 5 minutes three times in 1% (v/v) TBS-Tween, and incubated with the appropriate secondary horseradish peroxidase linked enzyme for 1 hour at room temperature. Again the membranes were washed for 5 minutes three times in 1% (v/v) TBS-Tween.

Membranes were developed by enhanced chemiluminescence (ECL) according to manufacturer's instructions (Amersham).

(viii) siRNA Assays
Transient Transfection of siRNA Oligos Using Oligofectaminutese

siRNAs were designed and obtained from Dharmacon and Qiagen. U373/CD14 cells were seeded at 5×10⁴ cells/ml in 6 well plates. Oligos were made up to a final concentration of 20 μM. Oligos were then diluted 1/10 in serum free medium. A 1/5 dilution of oligofecta-mine was made up using serum free media (SFM. Oligofecta-mine was added to siRNA and incubated for 20 minutes. Cells were washed once with 1 ml of SFM. 880 μl of SFM was then added to the cells. siRNA and oligofecta-mine mix was added to the cells. After 6 hours, 1 ml of media containing 20% FCS and 2×L glutamine was added to the cells. Cells were harvested after 48 hours. Samples were examined by western blot according to the protocol outlined above.

Transfection of siRNA Oligos Using the AMAXA System

Human PBMC cells are semi adherent and THP1 cells are a suspension cell line, therefore they are difficult to transfect by conventional lipid based transfection methods. Therefore the Amaxa system was used as it is capable of transfecting suspension cells. The cell line NUCLEOFECTOR Kit V™ (Lonza Cologne AG, Germany) was used for all siRNA transfections. 1×10⁶ cells/ml PBMC or THP1 cells were used per point. Between 0.5-3 μg of siRNA was added to the cells, then combined with 100 μl of NUCLEOFECTOR™ solution V and transferred to an Amaxa certified cuvette. Each sample was processed separately to avoid storing cells in NUCLEOFECTOR™ solution V for more than 15 minutes. The S-019 program was set on the NUCLEOFECTOR™, the sample was inserted and electroporated. An Amaxa pipette was used to transfer the sample into 500 μl of pre-warmed RPMI containing 10% FCS and 0.1% Penicillin, streptomycin for THP1 cells and IMDM containing 10% FCS and 0.1% Ciprofloxacin for PBMC. Cells were incubated at 37° C. for 72 hours. Cells were lysed directly in sample buffer as previously described and analysed by western blot as described above.

### (ix) Immunoprecipitation

### Antibody Precoupling

The relevant antibodies were precoupled with protein G sepharose beads, by incubating 15-30 μl of antibody with 40 μl of beads overnight with gentle rotation at 4° C.

### Immunoprecipitation

Cells were seeded at 2×10⁵ cells/ml in 10 cm dishes. The cells were grown for 24 hours in media containing 10% FCS. Transfections were carried out using GENEJUICE transfection reagent (Novagen) as previously described. 10 μg of DNA plasmid was transfected. The cells were harvested 24 hours post-transfection. The cells were washed 3 times in sterile PBS. 800 μl of high stringency lysis buffer (50 mM HEPES, pH 7.5, 100 mM NaCl, 1 mM EDTA, 10% glycerol (v/v), 1% NP-40 (v/v) containing 10 μg/ml PMSF, 30 μg/ml aprotinin and 1 μg/ml sodium orthovanadate) was added to the cells. The cells were removed from the 10 cm dish using a rubber cell scraper and transferred into a clean Eppendorf tube. Tubes were placed at 4° C. for 30 minutes with continuous gentle rotation. Samples were then centrifuged at 17,900×g for 10 minutes. Supernatants were removed and added to the relevant pre-coupled antibody. Samples were either incubated overnight at 4° C. or for 2 hours at room temperature. 50 μl of each lysate was retained to confirm expression of the protein of interest, this was added to sample buffer and boiled at 100° C. for 5 minutes. Following incubation the immune complexes were washed twice with 1 ml lysis buffer and once with ice cold PBS. All supernatant was removed and beads were resuspended in 30 μl of 5× sample buffer. The samples were boiled for 5 minutes and SDS-PAGE analysis was performed on the precipitated complexes as described previously.

### (x) Enzyme Linked Immunosorbant Assays (ELISA)

### Sample Preparation

BMDM were seeded at 1×10⁵ cells/ml in a 96 well plate. U373/CD14 cells were seeded at 2×10⁵ cells/ml and PBMC were seeded at 1×10⁶ cells/ml in 24 well plates. 24 hours later the cells were either left untreated or stimulated with the appropriate ligand for 24 hours. The supernatants were then removed to a new sterile 96 well plate or into sterile Eppendorf tubes and were assayed on that day or stored at −80° C. indefinitely.

### Plate Preparation

Capture antibody was diluted to the working concentration in PBS pH 7.4 (0.2 μm filtered). i.e.: human TNF-α (tumour necrosis factor alpha) working concentration of 4.0 μg/ml, human IL-6 working concentration of 2.0 μg/ml, human IL-1β working concentration of 4.0 μg/ml, human RANTES working concentration of 1.0 μg/ml, murine RANTES working concentration of 2.0 μg/ml, murine IL-6 working concentration of 2.0 μg/ml, murine TNF-α working concentration of 0.8 μg/ml.

A 96 well plate was immediately coated with 100 μl per well of the diluted capture antibody, the plate was sealed and incubated overnight at 4° C. The capture antibody was aspirated from each well, and the plate was washed with wash buffer (0.05% Tween 20 in PBS, pH 7.2-7.4), for a total of three washes. Complete removal of liquid at each step was essential for good performance. After the last wash, any remaining wash buffer was removed by inverting the plate and blotting it against clean paper towels. Te plate was then blocked by adding 300 μl of reagent diluent (1% BSA in PBS, pH 7.2-7.4) to each well. Plates were incubated at room temperature for a minimum of 1 hour. The reagent diluent was aspirated from each well, and each well was then washed with wash buffer for a total of three washes. After the last wash, any remaining wash buffer was removed by inverting the plate and blotting it against clean paper towels.

### Assay Procedure

The standards were prepared by diluting the recombinant protein in reagent diluent with a high standard concentration of 2000 pg/ml. A seven point standard curve using 2-fold serial dilutions in reagent diluent was then prepared. 100 μl of sample or standard in reagent diluent was added to each well. The plates were covered with an adhesive strip and incubated for 2 hour at room temperature. The samples and standards were then aspirated from each well, and each well was washed with wash buffer for a total of three washes. After the last wash, any remaining wash buffer was removed by inverting the plate and blotting it against clean paper towels. 100 μl of the working concentration of detection antibody, diluted in reagent diluent, was then added to each well. i.e. human TNF-α working concentration of 4.0 μg/ml; human IL-6 working concentration of 200 ng/ml; human IL-1β working concentration of 300 ng/ml; human RANTES working concentration of 10 ng/ml; murine RANTES working concentration of 400 ng/ml; murine IL-6 working concentration of 200 ng/ml; murine TNF-α working concentration of 150 ng/ml.

Once the detection antibody was added the plate was covered with a new adhesive strip and incubated for 2 hours at room temperature. The detection antibody was then aspirated from each well, and each well was washed with wash buffer for a total of three washes. After the last wash, any remaining wash buffer was removed by inverting the plate and blotting it against clean paper towels. 100 μl of the working dilution of streptavidin-HRP (1:200 dilution of streptavidin-HRP in 1% BSA-PBS) was added to each well. The plate was covered and incubated for 20 minutes at room temperature away from direct light. The streptavidin-HRP was then aspirated from each well, and each well was washed three times with wash buffer. After the last wash, any remaining wash buffer was removed by inverting the plate and blotting it against clean paper towels. 100 μl of substrate solution (A 1:1 mixture of Colour Reagent A (H₂0₂) and Colour Reagent B (tetramethybenzidine)) was then added to each well. Once the substrate solution was added the plates were incubated for 20 minutes at room temperature away from direct light. 50 μl of stop solution (2N H₂SO₄) was then added to each well. The plate was gently tapped to ensure thorough mixing. The optical density of each well was determined immediately, using a microplate reader set to 450 nm.

### Results

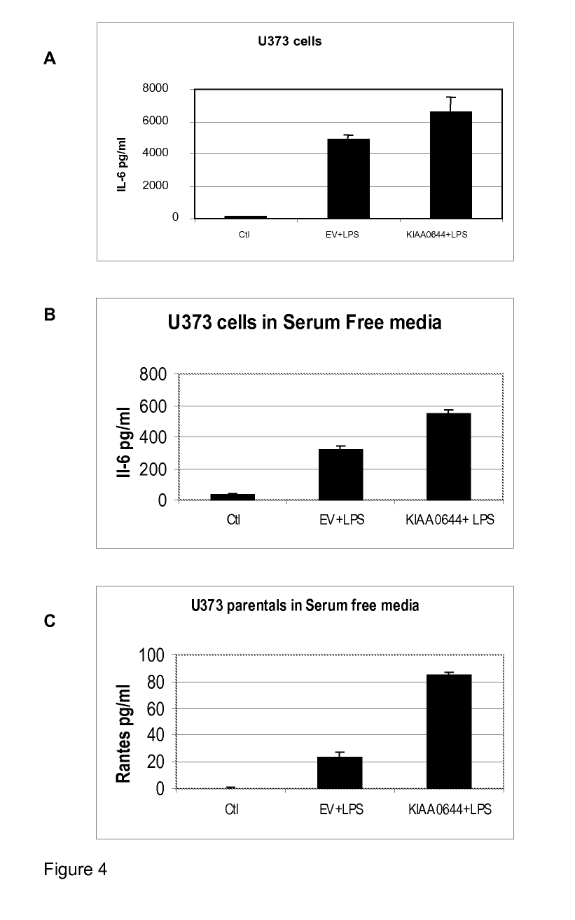

FIG. 4 shows the transient over-expression of KIAA0644 (the gene product encoding the TLR14 polypeptide) enhances IL-6 and RANTES production in U373 parental cells. U373 cells were seeded at a concentration of 2×10⁵ cells/ml in 6 well plates. After 24 hours 3 μg of empty vector (pcDNA3.1) or 3 μg of pcDNA-KIAA0644 was transfected into cells using GENEJUICE™. After 24 hours the media was changed to serum free media, with the results being shown in FIG. 4B and FIG. 4C. The cells providing the result shown in FIG. 4A were maintained in complete media. Cells were all stimulated with 100 ng/ml LPS for 24 hours. Supernatants were then removed from the cells and IL-6 (FIG. 4B) and RANTES (FIG. 4C) ELISAs were carried out. FIGS. 4 A, B and C therefore show the results of three independent experiments.

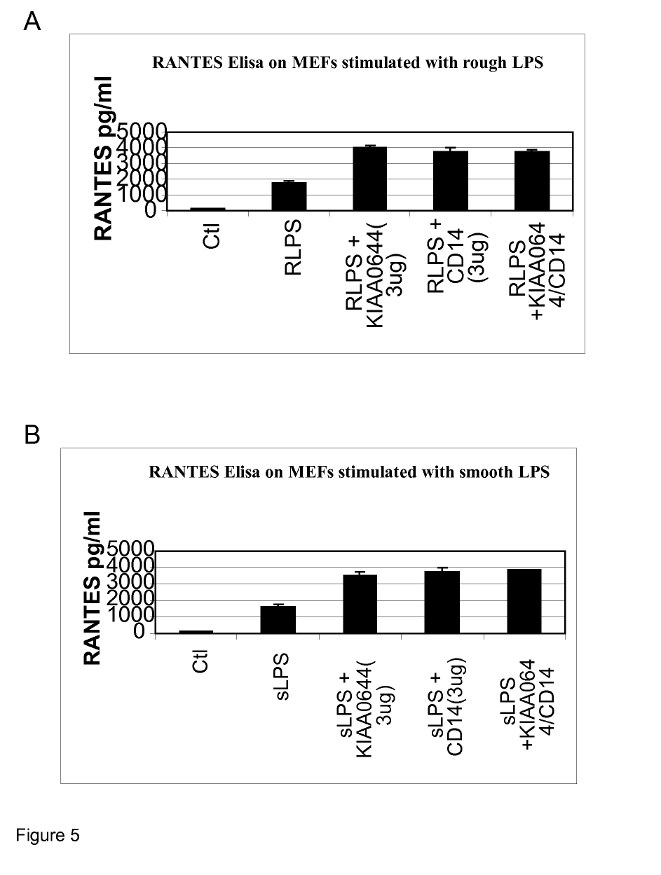

FIG. 5 shows the results of experiments involving the transient over-expression of KIAA0644 in MEF (mouse embryonic fibroblast) cells causes an increase in RANTES production in response to both rough (FIG. 5A) and smooth (FIG. 5B) LPS stimulation. MEF cells were seeded at 1×10⁵ cells/ml in 6 well plates. After 24 hours the cells were transfected with 3 μg of pcDNA 3.1 (Ctl), 3 μg of pcDNA-KIAA0644, 3 μg of pcDNA-CD14 or 1.5 μg of pcDNA-KIAA0644 together with 1.5 μg of pcDNA-CD14. After 48 hours, cells were stimulated with 100 ng/ml of rough or smooth LPS for an additional 24 hours. Supernatants were removed and RANTES ELISAS were carried out. The results are shown in FIG. 5A for stimulation with rough LPS and in FIG. 5B for stimulation with smooth LPS.

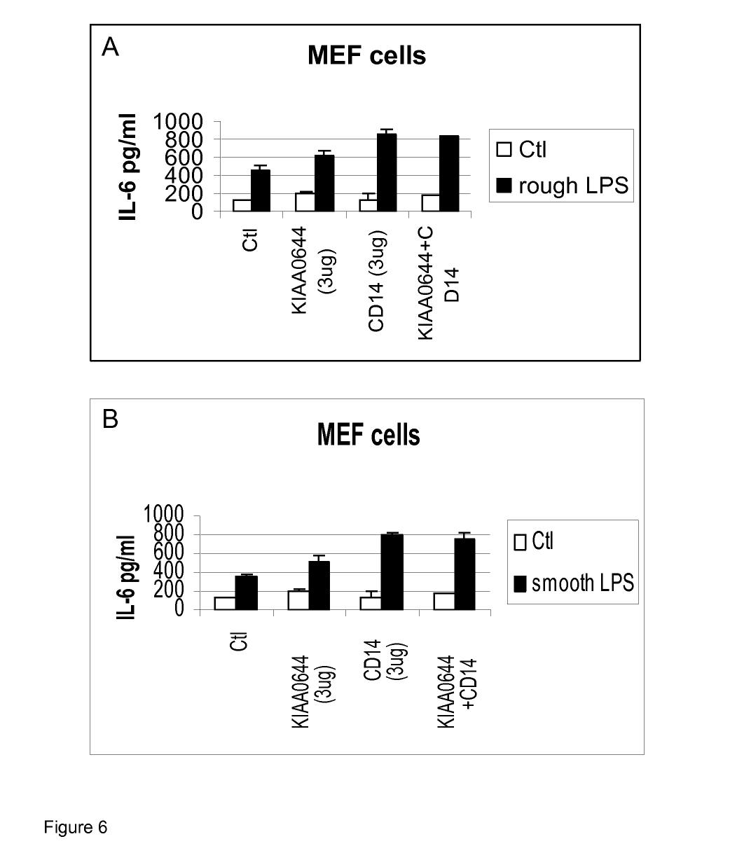

FIG. 6 shows the results of transient over-expression of KIAA0644 in MEF cells causes an increase in IL-6 production in response to both rough (FIG. 6A) and smooth (FIG. 6B) LPS. MEF cells were seeded at 1×10⁵ cells/ml in 6 well plates. After 24 hours 3 μg of pcDNA 3.1 (Ctl), 3 μg of plasmid expressing KIAA0644, 3 μg of plasmid expressing CD14 or both together were transfected into cells using GENEJUICE™. After 48 hours the cells were stimulated with 100 ng/ml of rough or smooth LPS for an additional 24 hours. Supernatants were removed and RANTES ELISAS were carried out. The results for stimulation with rough LPS are shown in FIG. 6A, while the results for stimulation with smooth LPS are shown in FIG. 6B.

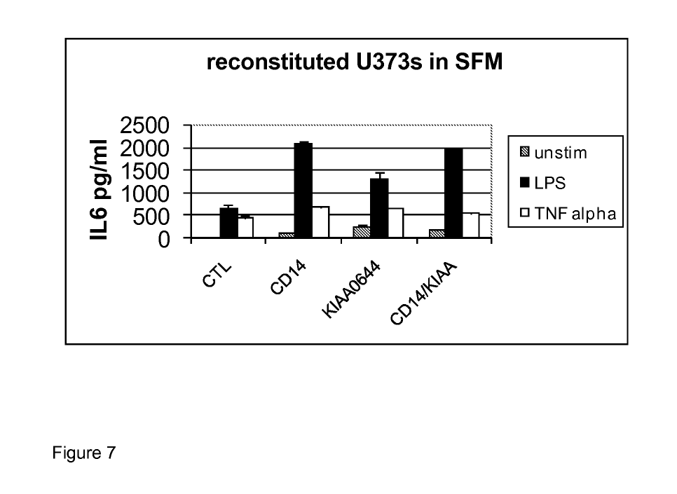

FIG. 7 shows that the reconstitution of U373 parental cells with KIAA0644 (TLR14) boosts the LPS signalling pathway, but not the TNF-α signalling pathway. U373 cells were seeded at a concentration of 1×10⁵ cells/ml in 6 well plates. After 24 hours 3 μg of empty vector (pcDNA3.1) or 3 μg of pcDNA-KIAA0644 or both together were transfected into cells using GENEJUICE™. After 48 hours the media was changed to serum free media. Cells were stimulated with 100 ng/ml LPS or 20 ng/ml TNF-α for 24 hours. Supernatants were then removed from the cells and an IL-6 ELISA was carried out.

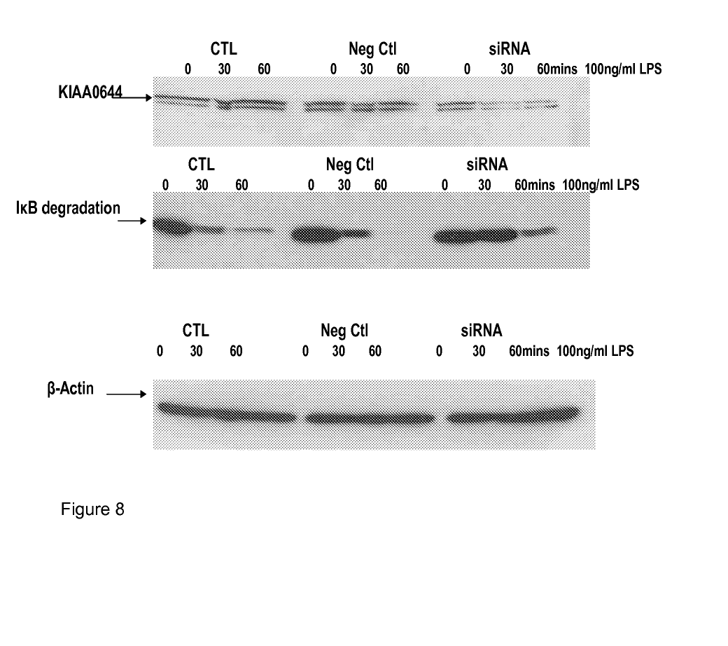

FIG. 8 shows the partial knockdown of KIAA0644 (TLR14) in U373/CD14 cells affects the LPS signalling pathway. U373/CD14 cells were set up at 5×10⁴ cells/ml in 6 well plates. After 24 hours the media was changed to serum free media. The control cells (CTL) were untransfected. siRNA from Qiagen was transfected at a concentration of 50 nM using oligofectamine. The negative control cells were transfected with a scrambled version of siRNA at a concentration of 50 nM. Cells were incubated for 72 hours before being stimulated with 100 ng/ml LPS for the time points indicated above. 60 μl of sample buffer was added directly to the cells. Samples were sonicated, resolved on a 10% SDS gels, transferred onto PVDF membrane, and immunoblotted for IκB and β-actin respectively at 0, 30 and 60 minutes.

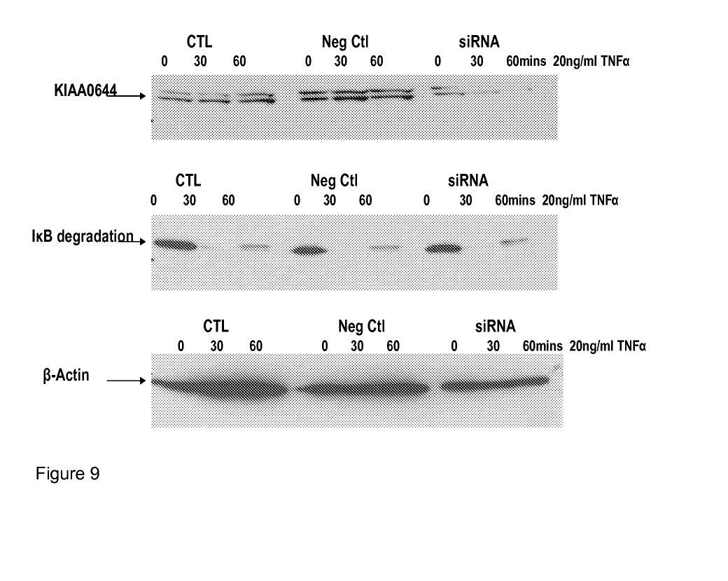

FIG. 9 shows that knockdown of KIAA0644 (TLR14) in U373/CD14 cells does not affect the TNF-α signalling pathway. U373/CD14 cells were set up at 5×10⁴ cells/ml in 6 well plates. After 24 hours the media was changed to serum free media. The control cells were untransfected. siRNA from Qiagen was transfected at a concentration of 50 nM using oligofectamine. The negative control cells were transfected with a scrambled version of siRNA at a concentration of 50 nM. Cells were incubated for 72 hours before being stimulated with 20 ng/ml TNF-α for the time points indicated above. 60 μl of sample buffer was added directly to the cells. Samples were sonicated, resolved on a 10% SDS gels, transferred onto PVDF membrane, and immunoblotted for IκB and β-actin respectively.

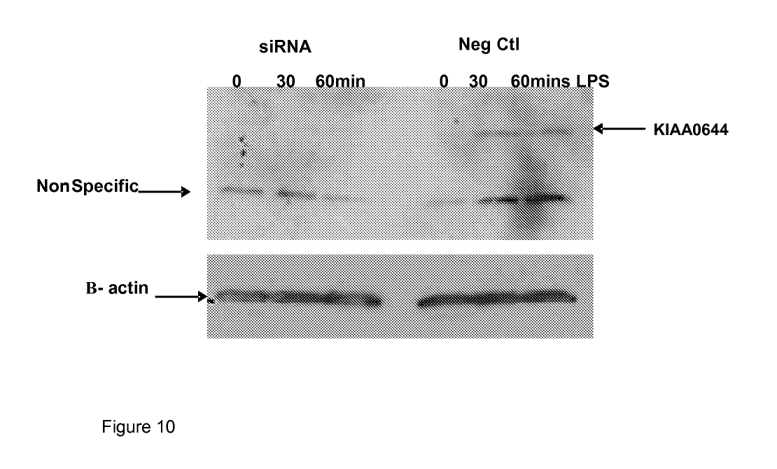

FIG. 10 shows that KIAA0644 (TLR14) can be knocked down using siRNA in human peripheral blood mononuclear cells (PBMCs). PBMCs were isolated from whole blood using a ficoll gradient. Cells were set up at a concentration of 2×10⁶ cells per point. siRNA from Qiagen or a scrambled negative control (Neg ctl) of the siRNA was transfected into the cells using the Amaxa program S-019 (Amaxa Biosystems, cologne, Germany). Following electroporation, the cells were added to complete IMDM media in 24 well plates and incubated at 37° C. for 72 hours. Samples were stimulated with 100 ng/ml LPS for 24 hours. Samples were harvested, lysed and sonicated in sample buffer, resolved on 10% SDS gels, transferred onto PVDF membrane, and immunoblotted with anti-KIAA0644 and R-actin respectively. A negative control (Neg Ctl) is also shown, this showing KIAA0644 expression in the absence of siRNA.

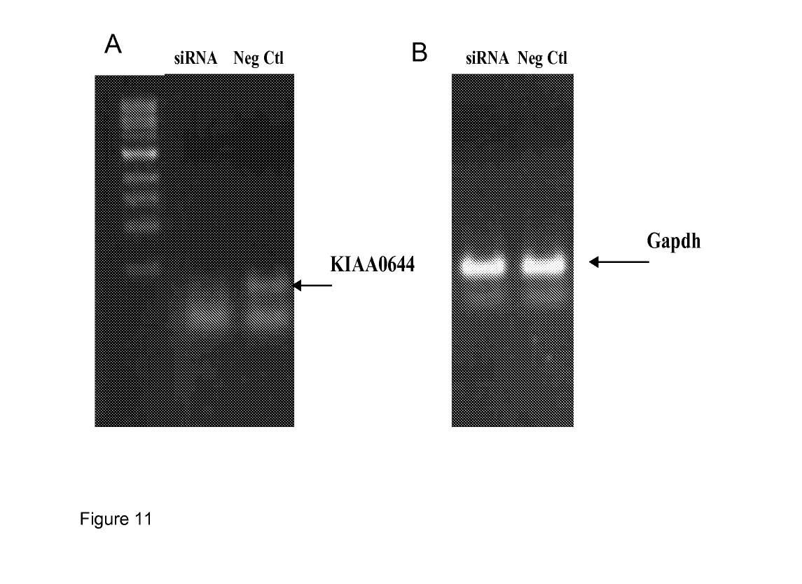

FIG. 11 shows RT-PCR experiments that confirm knockdown of KIAA0644 (TLR14) in human PBMC. Blood was obtained from a blood bank, PBMC were isolated using a ficoll gradient. Cells were set up at a concentration of 2×10⁶ cells per point. siRNA from Qiagen or a scrambled control of the siRNA was transfected into the cells using the Amaxa program S-019 (Amaxa Biosystems, cologne, Germany). Subsequently the samples were added to complete RMPI medium in 24 well plates and incubated at 37° C. for 72 hours. Cells were harvested and lysed in buffer R1. Primers specific to KIAA0644 were designed and RT-PCR was performed. The gel in FIG. 11A shows KIAA0644 (TLR14) expression, while FIG. 11B shows Gapdh expression. In each gel, a negative control (Neg Ctl) is shown.

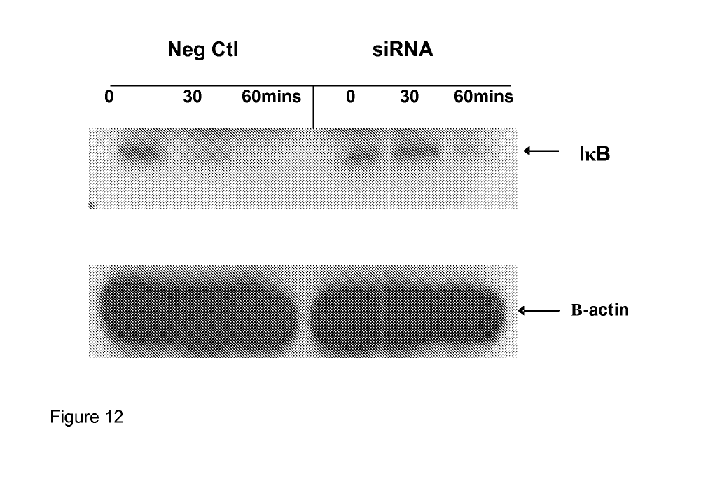

FIG. 12 shows the results of experimentation which shows that the knockdown of KIAA0644 (TLR14) in PBMC affects IκB degradation in response to LPS stimulation. PBMC were isolated from whole blood using a ficoll gradient. Cells were set up at a concentration of 2×10⁶ cells per point. siRNA from Qiagen or a scrambled control (Neg Ctl) of the siRNA was tranfected into the cells using the Amaxa program S-019. Following electroporation the samples were added to complete IMDM media in 24 well plates and incubated at 37° C. for 72 hours. Samples were stimulated with 100 ng/ml LPS for 24 hours. Samples were harvested, lysed and sonicated in sample buffer, resolved on a 10% SDS gels, transferred onto PVDF membrane, and immunoblotted with anti-IκB and β-actin respectively.

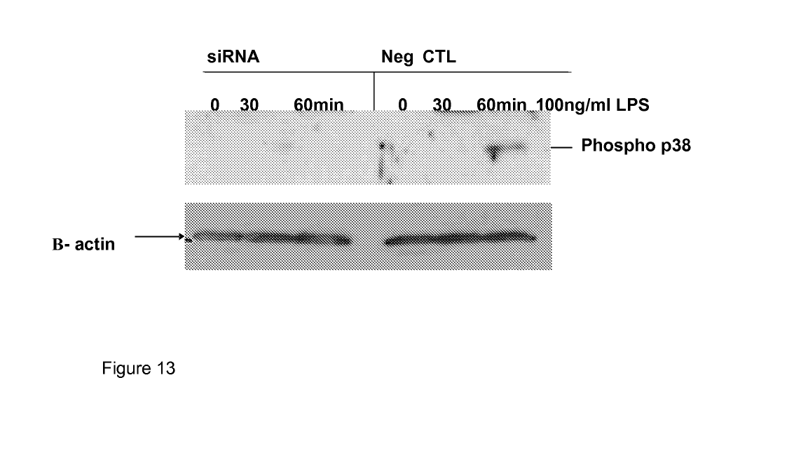

FIG. 13 shows the knockdown of KIAA0644 affects phosphorylation of p38 in response to LPS stimulation. PBMC were isolated from whole blood using a ficoll gradient. Cells were set up at a concentration of 2×10⁶ cells per point. siRNA from Qiagen or a scrambled control of the siRNA was tranfected into the cells using the AMAXA program S-019. Subsequently the samples were added to complete IMDM media in 24 well plates and incubated at 37° C. for 72 hours. Samples were stimulated with 100 ng/ml LPS for 24 hours. Samples were harvested, lysed and sonicated in sample buffer, resolved on 10% SDS gels, transferred onto PVDF membrane, and immunoblotted with anti-phospho p38 and β-actin respectively. A negative control (Neg CTL) is also shown, showing Phospho p38 MAPK expression.

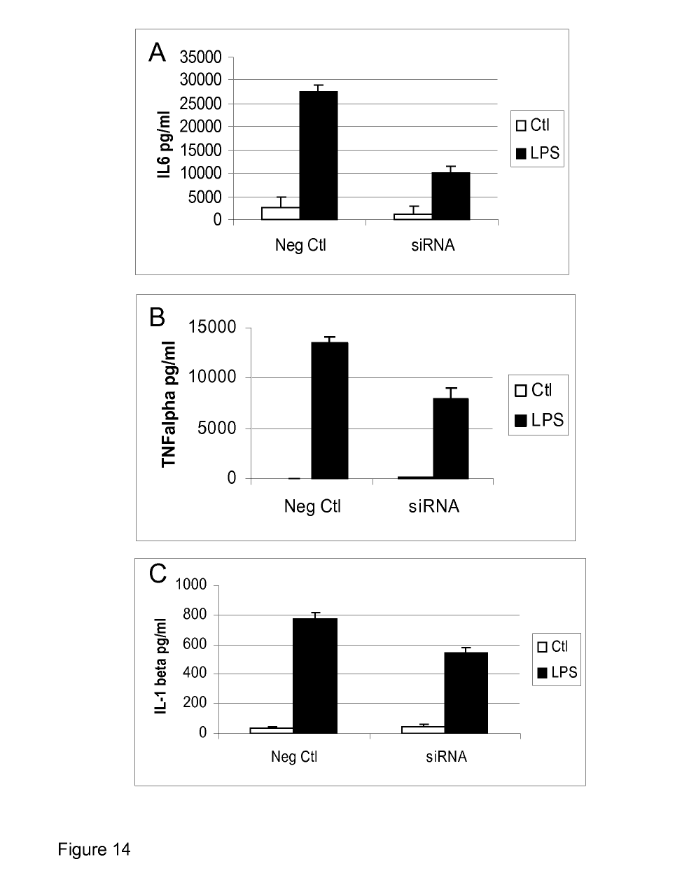

FIG. 14 shows 3 graphs showing that knockdown of KIAA0644 (TLR14) in PBMC causes a decrease in IL-6 (FIG. 14A), TNF-α (FIG. 14B) and IL-1β (FIG. 14C) release in response to LPS stimulation. PBMCs were isolated from whole blood using a ficoll gradient. Cells were set up at a concentration of 2×10⁶ cells per point. siRNA from QIAGEN or a scrambled control (Neg Ctl) of the siRNA was tranfected into the cells using the AMAXA program S-019 (Amaxa Biosystems, cologne, Germany). Following electroporation, the samples were added to complete IMDM media (Iscove's Modified Dulbecco's Medium) (Invitrogen, California, USA) in 24 well plates and incubated at 37° C. for 72 hours. Samples were stimulated with 100 ng/ml LPS for 24 hours. They were removed and centrifuged at 1000×g for 5 minutes. Supernatants were removed, TNF-α (FIG. 14B), IL-6 (FIG. 14A) and IL-1β (FIG. 14C) ELISAs were carried out.

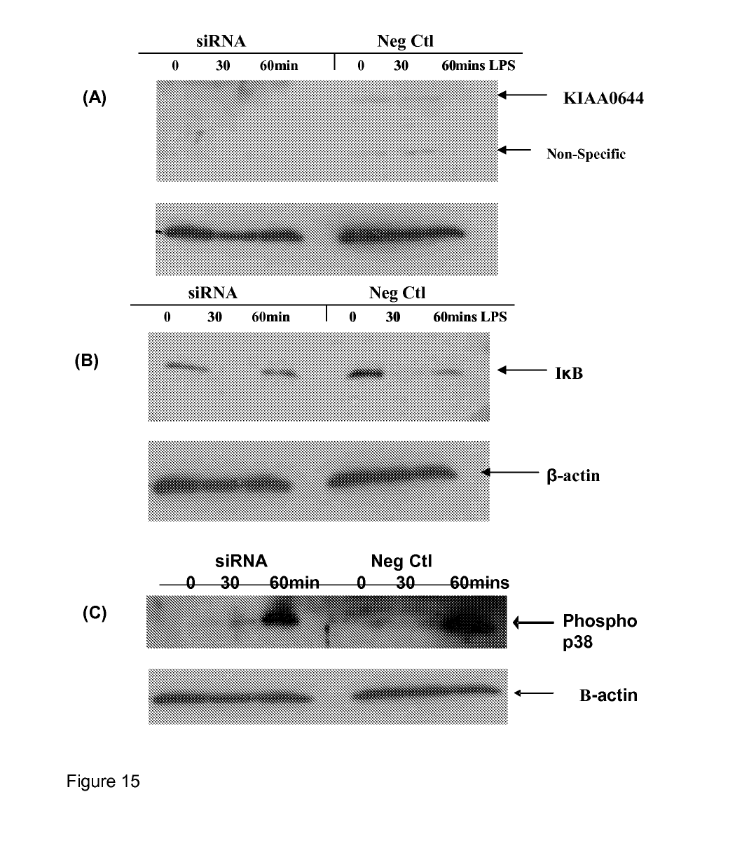

FIG. 15 shows the knockdown of KIAA0644 (TLR14) in PBMC does not affect the TNF-α signalling pathway. PBMC were isolated from whole blood using a ficoll gradient. Cells were set up at a concentration of 2×10⁶ cells per point. siRNA from Qiagen or a scrambled negative control (Neg Ctl) of the siRNA was transfected into the cells using the AMAXA program S-019 (Amaxa Biosystems, Cologne, Germany). Following electroporation the cells were added to complete IMDM media in 24 well plates and incubated at 37° C. for 72 hours. Samples were stimulated with 2 ng/ml TNF-α for 30 and 60 minutes. Samples were harvested, lysed and sonicated in sample buffer, resolved on a 10% SDS gels, transferred onto PVDF membrane. They were immunoblotted with anti-KIAA0644 and β-actin (FIG. 15 A), anti-IκB and β-actin (FIG. 15 B) and with anti-phospho p38 and β-actin (FIG. 15 C) respectively.

In summary, it can be determined that LPS-mediated activation of TLR4 can occur in the presence or absence of both CD14 and TLR14. However, the highest levels of TLR4 activation are shown when both TLR14 and CD14 are present. This suggests a role for CD14 in LPS-mediated TLR-4 activation and signalling, for example by CD14 binding LPS and trafficking it to TLR4.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention. Reference to any prior art in this specification is not, and should not be taken as, an acknowledgment or any form of suggestion that this prior art forms part of the common general knowledge in any country.

1. A method for the identification of a compound which modulates the dissociation or formation of a heterodimer formed between Toll-like Receptor 14 and CD14, said method comprising the steps of:
providing first and second cellular samples comprising CD14, Toll-like Receptor 4 and Toll-like Receptor 14,
contacting said first and second samples with a Toll-like Receptor 4 agonist,
contacting said first sample only with a candidate modulator agent under conditions permissive of binding of said agent to at least one of CD14 and Toll-like Receptor 14, and
monitoring the activation status of the Toll-like Receptor 4 receptor complex through a comparison of the level of downstream intracellular signalling between said first and second samples,
wherein a change in Toll-like Receptor 4 signalling between said first sample and said second sample identifies the candidate modulator agent as a modulator of the dissociation or formation of the heterodimer between CD14 and Toll-like Receptor 14.

2. (canceled)

3. A method for the identification of a compound which inhibits the binding of a Toll-like Receptor 4 agonist to CD14, said method comprising the steps of:
providing first and second cellular samples comprising CD14,
contacting said first and second samples with a Toll-like Receptor 4 agonist,
contacting said first sample only with a candidate modulator agent under conditions permissive of binding of said agent to CD14, and
monitoring the activation status of the Toll-like Receptor 4 receptor complex through a comparison of the level of downstream intracellular signalling between said first and second samples,
wherein a reduction in Toll-like Receptor 4 signalling between said first sample and said second sample identifies the candidate modulator agent as an inhibitor of the binding of a Toll-like Receptor 4 agonist to CD14.

4. The method of claim 1, wherein the TLR4 agonist is lipopolysacchande (LPS).

5. The method of claim 1, wherein the level of Toll-like Receptor 4 intracellular signalling is determined by monitoring markers indicative of Toll-like Receptor 4 activity selected from the group consisting of: NF-kappaB activation, and IRF3 protein activation.

6. A method for the identification of an agent which acts as an antagonist of Toll-like Receptor 4 activation and intracellular signalling, said method comprising the steps of:
providing first and second cellular samples containing Toll-like Receptor 14, Toll-like Receptor 4 and CD 14,
labelling the Toll-like Receptor 14 with a first fluorophore molecule and the CD14 with a second fluorophore molecule,
contacting said first and second samples with a Toll-like Receptor 4 agonist,
contacting said first sample only with a candidate modulator agent under conditions permissive of binding of Toll-like Receptor 4 and/or Toll-like Receptor 14, and
monitoring the interaction of CD14 and TLR14 to determine any dissociation which occurs in the presence of the Toll-like Receptor 4 agonist and the candidate agent by monitoring the fluorescence of the fluorophores,
wherein a decrease in the level of fluorescence is indicative of the candidate modulator agent not being an antagonist of Toll-like Receptor 4 activation and intracellular signalling.

7. (canceled)

8. A method for modulating Toll-like Receptor 4 activation and/or intracellular signaling, comprising:
contacting a cellular sample comprising CD14, Toll-like Receptor 4 and Toll-like Receptor 14 with an agent which has one or more of the following properties:
(i) inhibits the dissociation of a heterodimer complex formed between Toll-like Receptor 14 and CD14 when a Toll-like Receptor 4 agonist is bound to at least one of the Toll-like Receptor 4 or the CD14,
(ii) inhibits the formation of a heterodimer complex comprising Toll-like Receptor 14 and CD14,
(iii) inhibits the association of a Toll-like Receptor 4 agonist with CD14,
(iv) inhibits the transfer of a Toll-like Receptor 4 agonist which is bound to CD14 to Toll-like Receptor 14,
(v) inhibits the binding of a Toll-like Receptor 4 activating ligand to CD14, or
(vi) inhibits the transfer of a Toll-like Receptor 4 activating ligand from CD14 to Toll-like Receptor 4, thereby modulating Toll-like Receptor 4 activation and/or intracellular signalling.

9. The method of claim 8, wherein the agent is selected from the group consisting of: a protein, a peptide, a peptidomimetic, a small molecule compound, a nucleic acid, a polynucleotide, a polysaccharide, an oligopeptide, a carbohydrate, a lipid, naturally occurring compounds, and an antibody or a fragment thereof.

10.-38. (canceled)

39. A method for enhancing an immune response mediated by Toll-like Receptor 4, comprising administering to a subject in need thereof an effective amount of an agent which promotes the dissociation of a heterodimer formed between Toll-like Receptor 14 and CD14.

40. (canceled)

41. A method for the treatment and/or prophylaxis of a disease condition which is mediated by Toll-like Receptor 4 activation and/or Toll-like Receptor 4 intracellular signalling, the method comprising:
providing an effective amount of an agent which has one or more of the following properties:
(i) inhibits the dissociation of a heterodimer complex comprising Toll-like Receptor 14 and CD14,
(ii) inhibits the formation of a heterodimer complex comprising Toll-like Receptor 14 and CD14,
(iii) inhibits the association of a Toll-like Receptor 4 agonist with CD14,
(iv) inhibits the transfer of a Toll-like Receptor 4 agonist which is bound to CD14 to Toll-like Receptor 14,
(v) inhibits the binding of a Toll-like Receptor 4 activating ligand to CD14, or
(vi) inhibits the transfer of a Toll-like Receptor 4 activating ligand from CD14 to Toll-like Receptor 4, and
administering the same to a subject in need of such treatment.

42.-45. (canceled)

46. The method of claim 41, wherein the disease condition which is mediated by Toll-like Receptor 4 activation and/or Toll-like Receptor 4 intracellular signalling is an inflammatory condition, or a neurological condition or neurodegenerative disorder.

47.-48. (canceled)

49. A method as claimed in claim 46, wherein the neurodegenerative disorder or neurological condition is chosen from one or more of the group comprising: Parkinson's disease, Alzheimer's disease, Huntington's disease, amyotrophic lateral sclerosis (ALS), traumatic brain injury, spinal cord injury, multiple sclerosis, ischemia or ischemia-induced injury, stroke, or a neurodegenerative condition or disorder caused by a bacterial infection.

50. The method of claim 1, wherein the candidate modulator agent inhibits the dissociation or formation of the heterodimer between CD14 and Toll-like Receptor 14.

51. The method of claim 1, wherein the candidate modulator agent promotes the dissociation or formation of the heterodimer between CD14 and Toll-like Receptor 14.


SEQUENCE LISTING (8 sequences)

SEQ ID NO: 1 - protein, 811 aa, Homo sapiens
MEAARALRLLLVVCGCLALPPLAEPVCPERCDCQHPQHLLCTNRGLRVVPKTSSLPSPHDVLTYSLGGNFITNITAFDFHRLGQLRRLDLQYNQIRSLHPKTFEKLSRLEELYLGNNLLQALAPGTLAPLRKLRILYANGNEISRLSRGSFEGLESLVKLRLDGNALGALPDAVFAPLGNLLYLHLESNRIRFLGKNAFAQLGKLRFLNLSANELQPSLRHAATFAPLRSLSSLILSANSLQHLGPRIFQHLPRLGLLSLRGNQLTHLAPEAFWGLEALRELRLEGNRLSQLPTALLEPLHSLEALDLSGNELSALHPATFGHLGRLRELSLRNNALSALSGDIFAASPALYRLDLDGNGWTCDCRLRGLKRWMGDWHSQGRLLTVFVQCRHPPALRGKYLDYLDDQQLQNGSCADPSPSASLTADRRRQPLPTAAGEEMTPPAGLAEELPPQPQLQQQGRFLAGVAWDGAARELVGNRSALRLSRRGPGLQQPSPSVAAAAGPAPQSLDLHKKPQRGRPTRADPALAEPTPTASPGSAPSPAGDPWQRATKHRLGTEHQERAAQSDGGAGLPPLVSDPCDFNKFILCNLTVEAVGADSASVRWAVREHRSPRPLGGARFRLLFDRFGQQPKFHRFVYLPESSDSATLRELRGDTPYLVCVEGVLGGRVCPVAPRDHCAGLVTLPEAGSRGGVDYQLLTLALLTVNALLVLLALAAWASRWLRRKLRARRKGGAPVHVRHMYSTRRPLRSMGTGVSADFSGFQSHRPRTTVCALSEADLIEFPCDRFMDSAGGGAGGSLRREDRLLQRFAD

SEQ ID NO: 2 - protein, 809 aa, Mus musculus
MEGVGAVRFWLVVCGCLAFPPRAESVCPERCDCQHPQHLLCTNRGLRAVPKTSSLPSPQDVLTYSLGGNFITNITAFDFHRLGQLRRLDLQYNQIRSLHPKTFEKLSRLEELYLGNNLLQALVPGTLAPLRKLRILYANGNEIGRLSRGSFEGLESLVKLRLDGNVLGALPDAVFAPLGNLLYLHLESNRIRFLGKNAFSQLGKLRFLNLSANELQPSLRHAATFVPLRSLSTLILSANSLQHLGPRVFQHLPRLGLLSLSGNQLTHLAPEAFWGLEALRELRLEGNRLNQLPLTLLEPLHSLEALDLSGNELSALHPATFGHQGRLRELSLRDNALSALSGDIFAASPALYRLDLDGNGWTCDCRLRGLKRWMGNWHSQGRLLTVFVQCRHPPALRGKYLDYLDDQLLQNGSCVDPSPSPTAGSRQWPLPTSSEEGMTPPAGLSQELPLQPQPQPQQRGRLLPGVAWGGAAKELVGNRSALRLSRRGPGPHQGPSAAAPGSAPQSLDLHEKPGRGRHTRANLSQTEPTPTSEPASGTPSARDSWQRAAKQRLASEQQESAVQSVSGVGLPPLVSDPCDFNKFILCNLTVEAVSANSASVRWAVREHRSPRPQGGARFRLLFDRFGQQPKFQRFVYLPERSDSATLHELRGDTPYLVCVEGVLGGRVCPVAPRDHCAGLVTLPEAGGRGGVDYQLLTLVLLAVNALLVLLALAAWGSRWLRRKLRARRKGGAPVHVRHMYSTRRPLRSMGTGVSADFSGFQSHRPRTTVCALSEADLIEFPCDRFMDSTGGGTSGSLRREDHLLQRFAD

SEQ ID NO: 3 - protein, 799 aa, Homo sapiens
MELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 4 - protein, 374 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKETLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 5 - DNA, 24 bp, Artificial Sequence
  Description of Artificial Sequence Synthetic primer
gccttgcgcctcctgctcgtggtg

SEQ ID NO: 6 - DNA, 24 bp, Artificial Sequence
  Description of Artificial Sequence Synthetic primer
ccaccgcgagagcttctcgaaggt

SEQ ID NO: 7 - DNA, 21 bp, Artificial Sequence
  Description of Artificial Sequence Synthetic primer
gaacgggaagcttgtcatcaa

SEQ ID NO: 8 - DNA, 20 bp, Artificial Sequence
  Description of Artificial Sequence Synthetic primer
ctaagcagttggtggtgcag